(19) United States
(12) Patent Application Publication
(10) Pub. No.: US 2010/0222404 A1
(43) Pub. Date: Sep. 2, 2010

(54) INDAZOLE DERIVATIVE DIHYDROCHLORIDE

(75) Inventors: Seiji NAKANO, Tokyo (JP); Taisuke IWANAMI, Tokyo (JP); Yasuhiko MAEHARA, Tokyo (JP); Hitoshi KIDA, Tokyo (JP)

Correspondence Address: GREENBLUM & BERNSTEIN, P.L.C., 1950 ROLAND CLARKE PLACE, RESTON, VA, 20191, US

(73) Assignee: ASAHI KASEI PHARMA CORPORATION, Tokyo (JP)

(21) Appl. No.: 12/608,483

(22) Filed: Oct. 29, 2009

Related U.S. Application Data
(60) Provisional application No. 61/111,085, filed on Nov. 4, 2008.

(51) Int. Cl. A61K 31/416; C07D 231/56; A61P 13/00

(52) U.S. Cl. 514/406; 548/304.4

(57) ABSTRACT

There are provided (R)—N-[3-[2-[2-(3-methylindazol-6-yloxy)ethylamino]-1-hydroxyethyl]phenyl]methanesulfonamide dihydrochloride and a crystal thereof, and a crystal of the dihydrochloride salt having one or more major peaks at 2θ selected from the group consisting of approximately 12.8°, 21.8° and 25.0° in the powder X-ray diffraction spectrum.

### FIELD OF THE INVENTION

The present invention relates to (R)—N-[3-[2-[2-(3-methylindazol-6-yloxy)ethylamino]-1-hydroxyethyl]phenyl]methanesulfonamide dihydrochloride, which has β3 adrenergic receptor agonist activity and is useful as an active ingredient for medicines, and more particularly to crystals thereof.

### BACKGROUND OF THE INVENTION

WO 2003/035620 discloses a bicyclic compound having J33 adrenergic receptor agonist activity, which is represented by the following formula (I):

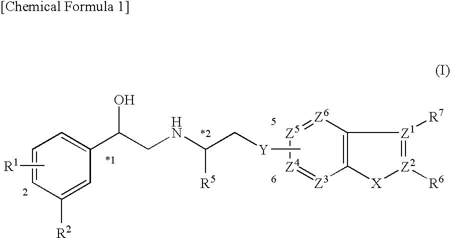

and the bicyclic compound has been found to be useful as an active ingredient for medicines for the prevention and/or treatment of, e.g., diabetes mellitus, obesity, hyperlipidemia and urinary incontinence.

WO 2003/035620 also discloses, as one of example compounds, (R)—N-[3-[2-[2-(3-methylindazol-6-yloxy)ethylamino]-1-hydroxyethyl]phenyl]methanesulfonamide hydrochloride (hereinafter, this salt may be referred to as “monohydrochloride salt” in the present specification).

### SUMMARY OF THE INVENTION

### Objects of the Invention

The present invention is to provide a novel acid addition salt of (R)—N-[3-[2-[2-(3-methylindazol-6-yloxy)ethylamino]-1-hydroxyethyl]phenyl]methanesulfonamide (hereinafter, also referred to as “compound 1” in the present specification) and particularly a crystal thereof having preferable properties as an active ingredient of medicines.

### The Solutions

As a result of extensive research for utilizing (R)—N-[3-[2-[2-(3-methylindazol-6-yloxy)ethylamino]-1-hydroxyethyl]phenyl]methanesulfonamide hydrochloride, “monohydrochloride salt” as a medicine, the present inventors recognized that the “monohydrochloride salt” has poor solubility, is difficult to prepare and shows significant variation in area under the concentration-time curve (AUC) when the “monohydrochloride salt” is orally administered.

The present inventors therefore studied various acid addition salts of “compound 1”, i.e. (R)—N-[3-[2-[2-(3-methylindazol-6-yloxy)ethylamino]-1-hydroxyethyl]phenyl]methanesulfonamide in an attempt to solve the above problems and unexpectedly found that, (R)—N-[3-[2-[2-(3-methylindazol-6-yloxy)ethylamino]-1-hydroxyethyl]phenyl]methanesulfonamide dihydrochloride (hereinafter, also referred to as “dihydrochloride salt” in the present specification) and, in particular, a crystal thereof show very high solubility in water. The inventors also established a process to easily produce the crystal, and therefore found the crystal is very useful as a drug substance. Furthermore, the inventors found that the AUC of the “dihydrochloride salt” exhibits less variation even if the salt is orally administered. The present invention was completed based on the findings described above.

Specifically, the present invention relates to the following;

(1) (R)—N-[3-[2-[2-(3-methylindazol-6-yloxy)ethylamino]-1-hydroxyethyl]-phenyl]methanesulfonamide dihydrochloride;

(2) a crystal of (R)—N-[3-[2-[2-(3-methylindazol-6-yloxy)ethylamino]-1-hydroxyethyl]phenyl]methanesulfonamide dihydrochloride;

(3) the crystal of (R)—N-[3-[2-[2-(3-methylindazol-6-yloxy)ethylamino]-1-hydroxyethyl]phenyl]methanesulfonamide dihydrochloride according to (2) above, wherein said crystal exhibits one or more major peaks at 2θ value selected from the group consisting of approximately 12.8°, 21.8° and 25.0° in the powder X-ray diffraction spectrum;

(4) the crystal of (R)—N-[3-[2-[2-(3-methylindazol-6-yloxy)ethylamino]-1-hydroxyethyl]phenyl]methanesulfonamide dihydrochloride according to (2) or (3) above, wherein said crystal exhibits major peaks at 2θ value of approximately 12.8°, 18.0°, 21.8° and 25.0° in the powder X-ray diffraction spectrum;

(5) the crystal of (R)—N-[3-[2-[2-(3-methylindazol-6-yloxy)ethylamino]-1-hydroxyethyl]phenyl]methanesulfonamide dihydrochloride according to any one of (2) to (4) above, wherein said crystal exhibits major absorption peaks around wavenumbers of 1646, 1341, 1286 and 1150 cm¹ in the infrared absorption spectrum;

(6) the crystal of (R)—N-[3-[2-[2-(3-methylindazol-6-yloxy)ethylamino]-1-hydroxyethyl]phenyl]methanesulfonamide dihydrochloride according to any one of (2) to (5) above, wherein said crystal exhibits a decomposition peak at approximately 241° C. in a differential scanning calorimetric analysis (heating rate: 10° C./min);

(7) a method for producing the crystal of (R)—N-[3-[2-[2-(3-methylindazol-6-yloxy)ethylamino]-1-hydroxyethyl]phenyl]methanesulfonamide dihydrochloride according to any one of (2) to (6) above, the method comprising preparing a solution by dissolving (R)—N-[3-[2-[2-(3-methylindazol-6-yloxy)ethylamino]-1-hydroxyethyl]phenyl]methanesulfonamide in a hydrochloric acid-containing solvent, and isolating crystals precipitated from the solution after, if necessary, mixing the solution with a poor solvent of certain type;

(8) a crystal of (R)—N-[3-[2-[2-(3-methylindazol-6-yloxy)ethylamino]-1-hydroxyethyl]phenyl]methanesulfonamide dihydrochloride which can be produced by the method for production according to (7) above;

(9) a pharmaceutical composition comprising as an active ingredient the dihydrochloride salt according to (1) above;

(10) a pharmaceutical composition comprising as an active ingredient the crystal according to any one of (2) to (6) and (8) above;

(11) a method for treating overactive bladder, comprising administering to a patient an effective amount of the dihydrochloride salt according to (1) above;

(12) a method for treating overactive bladder, comprising administering to a patient an effective amount of the crystal according to any one of (2) to (6) and (8) above;

(13) use of the dihydrochloride salt according to (1) above, for manufacturing a therapeutic agent for overactive bladder; and

(14) use of the crystal according to any one of (2) to (6) and (8) above, for manufacturing a therapeutic agent for overactive bladder.

The (R)—N-[3-[2-[2-(3-methylindazol-6-yloxy)ethylamino]-1-hydroxyethyl]phenyl]methanesulfonamide dihydrochloride provided by the present invention is characterized in that its solubility in water is extremely high as compared with “monohydrochloride salt” as described in Example 84 of PCT International Patent Application No. WO 2003/035620. Therefore, formulation is extremely easily achieved, allowing application of the salt in various forms of preparations.

In general, when pharmaceutical-compounds are industrially produced as raw materials for formulation, it is a very important aspect to produce the compounds in the form of powder, which is easier to handle in the formulation processes, from the viewpoint of supplying the preparation stably. Upon considering the size of production facilities required when powders of the pharmaceutical compounds are produced under GMP controls, in the case of a process of producing a “monohydrochloride salt” from the “compound 1” which is in a free form, it has been required to carry out the process under highly diluted conditions in order to avoid oil precipitation conditions or to avoid the compound from turning into a heterogeneous mass. On the other hand, if a process of producing a “dihydrochloride salt” from the “compound 1” which is in a free form is employed, there is no concern about the oil precipitation conditions or the formation of a heterogeneous mass, and therefore, the salt can be produced at higher concentrations as compared with the case of producing the “monohydrochloride salt”. Accordingly, the “dihydrochloride salt” of the present invention exhibits, particularly under the circumstances of carrying out the production in an industrial scale, there is obtained an excellent effect that the dihydrochloride salt can be produced at low cost and in an environment-friendly manner as compared with the “monohydrochloride salt”, because (1) the production process is completed with a small amount of solvent, and (2) the salt can be produced with small-scale production facilities.

A drug which is considered to exhibits variations in the AUC from individual to individual, may also induce possible significant adverse side effects in, e.g. the individuals who show greater AUC's than the value estimated from the average population. There is also a possibility that the efficiency of the drug therapy may not be sufficient in those individuals showing smaller AUC's than those the average population.

The “dihydrochloride salt” of the present invention exhibited less variation in the AUC as compared with the “monohydrochloride salt” when the salts were orally administered to dogs, which will be illustrated in Test Example 9 described below. Therefore, it is also recognized that the “dihydrochloride salt” would also exhibit less variation in the AUC when the salt is orally administered to human. Therefore, from the viewpoint of safety and effectiveness, the “dihydrochloride salt” is considered to be excellent as a pharmaceutical product as compared with the “monohydrochloride salt”.

### BRIEF DESCRIPTION OF THE DRAWINGS

FIG. 1 is a diagram illustrating a differential scanning calorimetric analysis spectrum of the “compound 1”;

FIG. 2 is a diagram illustrating a differential scanning calorimetric analysis spectrum of the “monohydrochloride salt”;

FIG. 3 is a diagram illustrating a differential scanning calorimetric analysis spectrum of the “dihydrochloride salt”;

FIG. 4 is a diagram illustrating a powder X-ray diffraction spectrum of the “compound 1”;

FIG. 5 is a diagram illustrating a powder X-ray diffraction spectrum of the “monohydrochloride salt”;

FIG. 6 is a diagram illustrating a powder X-ray diffraction spectrum of the “dihydrochloride salt”;

FIG. 7 is a diagram illustrating an infrared absorption spectrum of the “compound 1”;

FIG. 8 is a diagram illustrating an infrared absorption spectrum of the “monohydrochloride salt”; and

FIG. 9 is a diagram illustrating an infrared absorption spectrum of the “dihydrochloride salt”.

### DETAILED DESCRIPTION OF THE PREFERRED EMBODIMENTS

The “dihydrochloride salt” can be produced by dissolving “compound 1” in a mixture of (1) hydrochloric acid in an amount equal to or greater than an equivalent required to convert the “compound 1” to the “dihydrochloride salt”, (2) water in an amount sufficient to dissolve the “dihydrochloride salt”, and if necessary, (3) an organic solvent such as ethanol, thereby obtaining a solution of the “dihydrochloride salt”, and subsequently mixing this solution with a certain type of poor solvent, i.e. a solvent in which the “dihydrochloride salt” is difficult to dissolve, and isolating crystals precipitated therefrom. A suitable example of the method of mixing may be a method of adding a solvent in which the “dihydrochloride salt” is difficult to dissolve, to a hydrochloric acid solution of “dihydrochloride salt” or a hydrochloric acid-containing solution containing the “dihydrochloride salt”.

The lower limit amount of hydrochloric acid used when the “compound 1” is dissolved in hydrochloric acid, is preferably 2.0 equivalents or more with respect to the “compound 1”. The upper limit amount is not particularly limited as long as it is an amount exceeding 2.0 equivalents. The upper limit amount is preferably 2.5 equivalents or less, more preferably 3 equivalents or less, and particularly preferably 5 equivalents or less, though an embodiment of adding an amount exceeding 5 equivalents, for example, 10 equivalents, is also considered as one of preferred embodiments.

The concentration of hydrochloric acid used when the “compound 1” is dissolved in hydrochloric acid, is preferably 0.1 N or higher, and more preferably 1 N or higher. Furthermore, a concentration of 12 N or lower is preferred.

When the “compound 1” is dissolved in hydrochloric acid, the solvent which is used as needed is preferably an alcohol such as methanol, ethanol, 1-propanol or 2-propanol, or the like, or if necessary, these can also be used as a mixture. Among these, ethanol or 2-propanol is more preferred, and 2-propanol is particularly preferred. In another embodiment, ethanol is particularly preferred. Furthermore, these solvents may contain water at a proportion to the extent of not interrupting with the precipitation of crystals of the “dihydrochloride salt” in the subsequent operation.

In regard to the order of mixing the “compound 1”, hydrochloric acid, and the solvent which is used as needed, under the circumstance where the previously described solvent is used according to necessity in dissolving the “compound 1” in hydrochloric acid, there may be mentioned, for example, a method of mixing the “compound 1” and hydrochloric acid and then mixing the resultant with the solvent; a method of mixing the “compound 1” and the solvent and then mixing the resultant with hydrochloric acid; a method of mixing hydrochloric acid and the solvent and then mixing the resultant with the “compound 1”; and the like, and any of these methods may be used.

The amount of the solvent which is used as needed in dissolving the “compound 1” in hydrochloric acid, may vary depending on the concentration of hydrochloric acid and the amount of hydrochloric acid used in the conversion into salt, as well as the type of solvent and the like, while for example, in the case of obtaining the salt using 2 to 2.5 equivalents of 1 to 2 N hydrochloric acid, the amount of the solvent used for 1 g of the “compound 1” is 0 to 20 ml, preferably 0 to 10 ml, and more preferably 0 to 5 ml.

When the “compound 1” is dissolved, the solution may be heated if necessary, and the temperature of the solution during the process of dissolution may be 0° C. to the reflux temperature of the solvent, and preferably from room temperature to the reflux temperature of the solvent.

In regard to the poor solvent of certain type, that is, the solvent in which the “dihydrochloride salt” is difficult to dissolve, which is intended to be mixed as necessary with a hydrochloric acid-containing solvent containing the “dihydrochloride salt”, for the purpose of precipitating crystals of the dihydrochloride salt, the same solvents as those used for producing the solution of the “dihydrochloride salt” mentioned above may be mentioned as examples. The amount of the poor solvent used may vary depending on the concentration of hydrochloric acid and the amount of hydrochloric acid used in the conversion into salt, as well as the type of solvent and the like, while for example, in the case of obtaining the salt using 2 to 2.5 equivalents of 1 to 2 N hydrochloric acid, the lower limit amount of the solvent used for 1 g of the “compound 1” is preferably 1 ml or more, more preferably 10 ml or more, and particularly preferably 20 ml or more. The upper limit amount is preferably 500 ml or less, more preferably 200 ml or less, and particularly preferably 100 ml or less.

The temperature at the time of adding the solvent in which the “dihydrochloride salt” is difficult to dissolve, is not particularly limited as long as it is an appropriate temperature in the range of 0° C. to the boiling temperature of the solvent, while it is preferable to add the solvent at a temperature in the range of room temperature to the reflux temperature of the solvent.

The method of mixing a solution of the “dihydrochloride salt” and a solvent in which the “dihydrochloride salt” is difficult to dissolve, is not particularly limited, and usually, the solvent in which the “dihydrochloride salt” is difficult to dissolve can be added in one time in the state in which the solution of the “dihydrochloride salt” is being stirred. However, the addition may be achieved in several divided portions, or may be achieved continuously over time by a method such as dropwise addition.

Upon precipitating the crystals, a method of cooling a solution obtained after mixing the solution of the “dihydrochloride salt” and the solvent in which the “dihydrochloride salt is difficult to dissolve, may be mentioned as a preferred embodiment. Furthermore, according to another preferred embodiment, a method of adding a small amount of crystals of the “dihydrochloride salt” that have been obtained previously, as seed crystals, to a solution obtained after adding the solvent in which the “dihydrochloride salt” is difficult to dissolve, may also be used. It is also acceptable to use these methods in combination. Examples of the method of cooling include a method of cooling rapidly, a method of cooling stepwise, a method of cooling slowly over time, a method of cooling naturally, and the like, while more preferred examples include a method of cooling stepwise, a method of cooling slowly over time, a method of cooling naturally, and the like.

The method of isolating precipitated crystals may be achieved by known methods such as filtration and decantation, and usually, it is preferable to isolate the crystals by filtration. Isolation of crystals can be carried out immediately after adding hydrochloric acid, and it is preferable to carry out the isolation after the precipitation of crystals has entered a steady state.

Upon collecting the precipitated crystals, it is preferable to collect the crystals after cooling the solution in which the precipitation of the crystals has entered a steady state, from the viewpoint of the yield of obtainable crystals or the like. Examples of the method of cooling include a method of cooling rapidly, a method of cooling stepwise, a method of cooling slowly over time, a method of cooling naturally, and the like, and a method of cooling stepwise, a method of cooling slowly over time, a method of cooling naturally, and the like are more preferred. The temperature for cooling is usually preferably 0° C. to 20° C., and more preferably 0° C. to 10° C.

After isolating the crystals by filtration, the crystals can be washed with a solvent. This operation is effective as an operation for removing impurities. Examples of the solvent used in washing include a solvent used in the dissolution of the compound 1, for example, ethanol, 2-propanol or water, or mixtures thereof. Examples of the method of washing include a method of rinsing the crystals on the filter with a solvent, and a method of introducing the crystals into a solvent to obtain a suspension, sufficiently stirring this suspension, and then collecting crystals again by filtration. It is also effective to carry out both of the two methods of washing.

The collected crystals can be dried by conventionally adopted methods of drying such as, for example, drying under reduced pressure, drying under elevated temperature and reduced pressure, drying by air blowing under elevated temperature, and by air drying.

Among the production methods described above, a preferred example may be a method of heating a mixture of the “compound 1” and 2-propanol to 60° C. to 80° C. to suspend the “compound 1”, adding hydrochloric acid to this suspension under stirring in an amount of 2 to 2.2 equivalents with respect to the “compound 1” to thereby completely dissolving the “compound 1” and to obtain a clear solution, subsequently cooling the solution naturally to room temperature, and further cooling the solution as necessary to obtain crystals. Furthermore, if necessary, a small amount of the previously obtained crystals of the “dihydrochloride salt” may be added to accelerate crystallization at an appropriate stage, for example, during the cooling or after the completion of cooling.

In general, when a medicinal compound is industrially produced as a raw material for formulation, it is a very important aspect to produce the compound in the form of powder, which is easier to handle in the formulation processes, from the viewpoint of supplying the preparation stably. Upon considering the size of production facilities required when a powder of the medicinal compound is produced under GMP controls, in the case of a crystallization process of producing “monohydrochloride salt” from “compound 1” which is in a free form, it has been required to carry out the process under highly diluted conditions in order to avoid the oil precipitation conditions or the compound turning into a heterogeneous mass. In contrast, in the case of a crystallization process of producing “dihydrochloride salt” from “compound 1” which is in a free form, there is less concern for facing the oil precipitation conditions or obtaining a heterogeneous mass, and therefore, the salt can be produced at a higher concentration of about three-fold the concentration used in the case of producing the “monohydrochloride salt”. Accordingly, the “dihydrochloride salt” of the present invention, particularly under the circumstances of carrying out the production in an industrial scale, can be produced by using about one-third of the solvent as compared to the amount of solvent to be required in the case of the “monohydrochloride salt”, and therefore, a production of about a three-fold amount of products can be achieved with production facilities of the same scale. Thus, there is obtained an excellent effect that the dihydrochloride salt can be produced at low cost and in an environment-friendly manner.

The “compound 1” in a free form that is used in the production of the “dihydrochloride salt”, can be produced by deprotecting all of the benzyl groups in (R)—N-benzyl-N-[3-[2-N′-benzyl-2-(1-benzyl-3-methylindazol-6-yloxy)ethylamino]-1-hydroxyethyl]phenyl]methanesulfonamide in a stepwise manner or all in a single time. The method of deprotection may be, for example, a method based on a hydrogenolysis reaction or the like. Specifically, the hydrogenolysis reaction may be carried out, for example, in a solvent [such as ethers (such as tetrahydrofuran, dioxane, dimethoxyethane or diethyl ether), alcohols (such as methanol or ethanol), benzene analogs (such as benzene or toluene), ketones (such as acetone or methyl ethyl ketone), nitriles (such as acetonitrile), amides (such as dimethylformamide), esters (such as ethyl acetate), water or solvent mixtures of two or more thereof], in the presence of a catalyst (such as palladium-carbon powder, platinum oxide (PtO₂) or activated nickel), and in the presence of a hydrogen source such as hydrogen gas under normal pressure or increased pressure, ammonium formate, or hydrazine hydrate, at a temperature of −10° C. to 60° C. During the reaction, it is preferable to add a mineral acid such as hydrochloric acid, or an organic acid such as acetic acid, into the reaction solution.

(R)—N-benzyl-N-[3-[2-N′-benzyl-2-(1-benzyl-3-methylindazol-6-yloxy)ethylamino]-1-hydroxyethyl]phenyl]methanesulfonamide, which is used in the production of the “compound 1” in a free form, can be produced according to the method described in Example 84 (Process B) of WO 2003/035620 (the disclosure of which is incorporated herein by reference).

In another method for producing the “compound 1” in a free form, the compound may also be produced based on the reaction pathways represented in Schemes 1 and 2 as illustrated below.

For example, the “compound 1” of the present invention can be produced by modifying or converting the substituents of a compound which serves as a precursor of the “compound 1”, through a single reaction or a combination of multiple reactions that are described in the general literatures in chemistry. Furthermore, the method that will be described later is described, unless particularly stated otherwise, for the convenience, to make use of a precursor compound (or an intermediate) in a free form; however, under certain circumstances, the compound can be produced using a salt of the precursor compound (or the intermediate) in a free form.

In regard to the respective reactions, the reaction time is not particularly limited, and since the progress of reaction can be easily traced by those analysis techniques mentioned below, it is favorable to terminate the reaction at a time point which gives the maximum yield of the target product. In the schemes 1 and 2 as illustrated below, the term “STEP” means a process, and the expression “STEP 1-1” means, for example, process 1-1. Furthermore, in the schemes 1 and 2 as illustrated below, the term “Compound 1” means the “compound 1.”

Examples of protective groups as used herein include protective groups for indazole (—NH—), hydroxyl (—OH), methanesulfonamide (—NHSO₂Me) and amino (—NH— or —NH₂).

Examples of protective groups for indazole (—NH—) include trityl, benzyl, methylbenzyl, chlorobenzyl, dichlorobenzyl, fluorobenzyl, trifluoromethylbenzyl, nitrobenzyl, methoxyphenyl, N-methylaminobenzyl, N,N-dimethylaminobenzyl, phenacyl, acetyl, trifluoroacetyl, pivaloyl, benzoyl, methoxycarbonyl, ethoxycarbonyl, allyloxycarbonyl (Alloc), 2,2,2-trichloroethoxycarbonyl, benzyloxycarbonyl (Cbz), tert-butoxycarbonyl (Boc), 1-methyl-1-(4-biphenyl)ethoxycarbonyl (Bpoc), 9-fluorenylmethoxycarbonyl, N,N-dimethylsulfonyl, methanesulfonyl, benzenesulfonyl, p-toluenesulfonyl, mesitylenesulfonyl, p-methoxyphenylsulfonyl, tetrahydropyranyl (THP), tetrahydrofuryl, allyl, methoxymethyl (MOM), methoxyethoxymethyl (MEM), benzyloxymethyl (BOM) and 2-(trimethylsilyl)ethoxymethyl (SEM).

Examples of protective groups for hydroxyl (—OH) include C₁-C₄ alkyl, C₂-C₄ alkenyl, C₁-C₄ alkyl substituted with C₁-C₄ alkoxy, C₁-C₄ alkyl substituted with 1 to 3 halogen atoms, silyl group substituted with three identical or different C₁-C₄ alkyl or phenyl, tetrahydropyranyl, tetrahydrofuryl, propargyl and trimethylsilylethyl. Specific examples include methyl, ethyl, tert-butyl, allyl, methoxymethyl (MOM), methoxyethoxymethyl (MEM), trichloroethyl, phenyl, methylphenyl, chlorophenyl, benzyl, methylbenzyl, chlorobenzyl, dichlorobenzyl, fluorobenzyl, trifluoromethylbenzyl, nitrobenzyl, methoxyphenyl, N-methylaminobenzyl, N,N-dimethylaminobenzyl, phenacyl, trityl, 1-ethoxyethyl (EE), tetrahydropyranyl (THP), tetrahydrofuryl, propargyl, trimethylsilyl (TMS), triethylsilyl (TES), text-butyldimethylsilyl (TBDMS), tert-butyldiphenylsilyl (TBDPS), acetyl (Ac), pivaloyl, benzoyl, allyloxycarbonyl (Alloc) and 2,2,2-trichloroethoxycarbonyl (Troc).

Examples of protective groups for methanesulfonamide group (—NHSO₂Me) include methoxycarbonyl, ethoxycarbonyl, tert-butoxycarbonyl (Boc), benzyl, methylbenzyl, chlorobenzyl, dichlorobenzyl, fluorobenzyl, trifluoromethylbenzyl, nitrobenzyl, methoxyphenyl, N-methylaminobenzyl, N,N-dimethylaminobenzyl, tert-butyl, diphenylmethyl and methoxyphenyl.

Examples of protective groups for amino (—NH— or —NH₂) include benzyl, methylbenzyl, chlorobenzyl, dichlorobenzyl, fluorobenzyl, trifluoromethylbenzyl, nitrobenzyl, methoxyphenyl, N-methylaminobenzyl, N,N-dimethylaminobenzyl, phenacyl, acetyl, trifluoroacetyl, pivaloyl, benzoyl, allyloxycarbonyl, 2,2,2-trichloroethoxycarboyl, benzyloxycarbonyl, tert-butoxycarbonyl (Boc), 1-methyl-1-(4-biphenyl)ethoxycarbonyl (Bpoc), 9-fluorenylmethoxycarbonyl, 2-nitrobenzenesulfonyl, 4-nitrobenzenesulfonyl, 2,4-dinitrobenzenesulfonyl, benzyloxymethyl (BOM) and 2-(trimethylsilyl)ethoxymethyl (SEM).

The protected compound can be converted to the desired compound by removing the protective groups in the middle of the production process or at the final step, simultaneously with the production or in succession. The protection/deprotection reactions may be carried out according to known methods, for example, the methods described in Protective Groups in Organic Synthesis, published by John Wiley and Sons (printed in 2007), while the reactions can be carried out by, for example, the methods listed in the following (1) to (3).

(1) A deprotection reaction under acidic conditions can be carried out, for example, in an inert solvent in the presence of organic acids, Lewis acids or inorganic acids, or in a mixture of these, at a temperature of −10° C. to 100° C. The amount of use of such an acid is preferably 1-fold the molar amount or a large excess, and a method of adding, as an additive, e.g., ethanethiol or 1,2-ethanedithiol may also be adopted.

Examples of such inert solvents include dichloromethane, chloroform, 1,4-dioxane, ethyl acetate, methyl tert-butyl ether, tetrahydrofuran and anisole. Examples of such organic acids include acetic acid, trifluoroacetic acid, methanesulfonic acid and p-toluenesulfonic acid. Examples of such Lewis acids include boron tribromide, boron trifluoride, aluminum bromide and aluminum chloride. Examples of such inorganic acids include hydrochloric acid, hydrogen chloride-1,4-dioxane, hydrogen chloride-ethyl acetate, hydrobromic acid and sulfuric acid. Such organic acids, Lewis acids, inorganic acids or mixtures thereof include hydrogen bromide and acetic acid.

(2) A deprotection reaction based on hydrogenolysis can be carried out, for example, in an inert solvent, with 0.1 to 300% by weight of a catalyst added therein, in the presence of a hydrogen source such as hydrogen gas under normal pressure or increased pressure, ammonium formate, or hydrazine hydrate, at a temperature of −10° C. to 70° C. The reaction can also be carried out by further adding an inorganic acid to the reaction liquid in an amount of 0.05-fold the molar amount to a large excess.

Examples of such inert solvents include ethers such as tetrahydrofuran, dioxane, dimethoxyethane and diethyl ether; alcohols such as methanol and ethanol, benzene analogues such as benzene and toluene; ketones such as acetone and methyl ethyl ketone; nitriles such as acetonitrile; amides such as dimethylformamide; esters such as ethyl acetate; water, and acetic acid. Such solvents can be used singly, or a mixture thereof may be used. Examples of such catalysts include palladium-carbon powder, platinum oxide (PtO₂) and activated nickel. Examples of such inorganic acids include hydrochloric acid and sulfuric acid.

(3) A deprotection reaction for a silyl group can be carried out, for example, in an organic solvent that is miscible with water, by using, for example, fluoride ions, at a temperature of −10° C. to 60° C.

Examples of such organic solvents include tetrahydrofuran, acetic acid and acetonitrile. The fluoride ions may be generated, for example, using tetra-n-butylammonium fluoride, hydrofluoric acid, a hydrogen fluoride-pyridine complex and a hydrogen fluoride triethylamine complex.

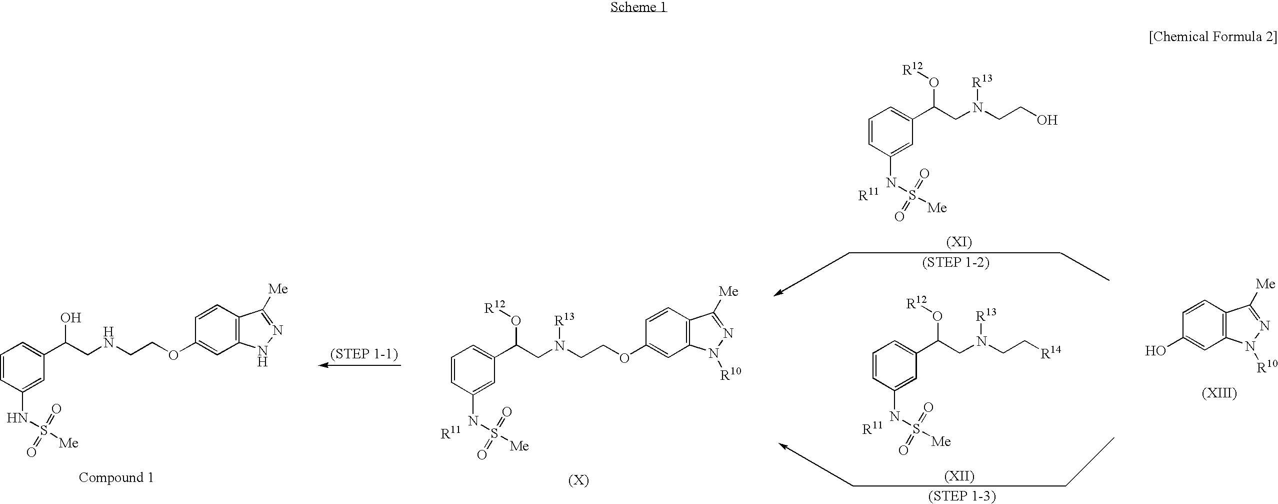

In the respective formulas in Scheme 1, R¹⁰ is a hydrogen atom or a protective group for indazole as described above, which is preferably a benzyl, tert-butoxycarbonyl or tetrahydropyranyl group; R¹¹ is a hydrogen atom or a protective group for methanesulfonamide as described above, which is preferably a benzyl or tert-butoxycarbonyl group; R¹² is a hydrogen atom or a protective group for hydroxyl as described above, which is preferably a triethylsilyl or tent-butyldimethylsilyl group; R¹³ is a hydrogen atom or a protective group for amino as described above, which is preferably a benzyl or tert-butoxycarbonyl group; and R¹⁴ represents a leaving group, and examples thereof include chlorine atom, bromine atom, iodine atom, p-toluenesulfonyloxy and methanesulfonyloxy, preferably bromine atom. Preferred combinations of R¹⁰, R¹¹, R¹² and R¹³ include ones of: R¹⁰ (benzyl), R¹¹ (benzyl), R¹² (triethylsilyl) and R¹³ (benzyl); R¹⁰ (tert-butoxycarbonyl), R¹¹ (tert-butoxycarbonyl), R¹² (triethylsilyl) and R¹³ (tert-butoxycarbonyl); and R¹⁰ (tetrahydropyranyl), R¹¹ (tert-butoxycarbonyl), R¹² (triethylsilyl) and R¹³ (text-butoxycarbonyl).

Process 1-1 (STEP 1-1)

A compound represented by “Compound 1” can be produced by subjecting a compound represented by the formula (X) to a deprotection reaction according to a known method, for example, a method described in Protective Groups in Organic Synthesis, published by John Wiley and Sons (printed in 2007). As a suitable example, it is preferable to perform the deprotection reaction under acidic conditions as described above, or it is preferable to utilize the deprotection reaction based on hydrogenolysis as described above alone, or to utilize these deprotection reactions in combination. At any rate, it is desirable to select an appropriate deprotection reaction for each of the various protective groups present in the compound represented by the formula (X),

Process 1-2 (STEP 1-2)

The compound represented by the formula (X) can be obtained by allowing a compound represented by the formula (XI) to react with a compound represented by the formula (XIII) in the presence of any phosphines and any azo compounds in an inert solvent.

In regard to the inert solvent, ethers such as diethyl ether, tetrahydrofuran or dimethoxyethane; halogenated solvents such as methylene chloride; benzene analogues such as benzene, toluene or xylene; or the like may be used singly, or a mixture of these may be used. Among them, toluene is preferred. The phosphine may be triphenylphosphine, tributylphosphine or the like, and triphenylphosphine is preferred. The azo compound may be diethyl azodicarboxylate, diisopropyl azodicarboxylate, N,N,N′,N′-tetramethylazodicarboxamide, 1,1′-(azodicarbonyl)dipiperidine, N,N,N′,N′-tetraisopropylcarboxamide, or the like, and N,N,N′,N′-tetramethylazodicarboxamide is preferred.

The amount of use of the phosphine may be 1- to 10-fold the molar amount, and preferably 1.5- to 5-fold the molar amount, of the compound represented by the formula (XI) or the compound represented by the formula (XIII). The amount of use of the azo compound may be 1- to 10-fold the molar amount, and preferably 1.5- to 5-fold the molar amount, of the compound represented by the formula (XI) or the compound represented by the formula (XIII). The molar ratio of the compound represented by the formula (XI) and the compound represented by the formula (XIII) may be such that compound represented by formula (XI)/compound represented by formula (XIII)=0.25 to 4. The reaction temperature may be from −20° C. to the reflux temperature, and is preferably 0° C. to 40° C. The reaction time may be from 0.1 hour to 48 hours, and preferably from 0.1 to 12 hours.

Process 1-3 (STEP 1-3)

A compound represented by the formula (X) can be obtained by allowing a compound represented by the formula (XII) to react with a compound represented by the formula (XIII) in an inert solvent in the presence of a base added thereto.

In regard to the inert solvent, water, alcohols such as methanol or ethanol, or N,N-dimethylformamide, tetrahydrofuran, 1,4-dioxane, acetone, 2-butanone, dimethylsulfoxide, acetonitrile or the like may be used singly, or a mixture of these may be used. However, water, N,N-dimethylformamide or acetone is preferred. Examples of the base include alkali metal compounds such as potassium carbonate, sodium carbonate, cesium carbonate, sodium hydrogen carbonate, potassium hydroxide, sodium hydroxide, sodium methoxide, and potassium t-butoxide; and organic tertiary amines such as pyridine, 4-dimethylaminopyridine, 1,8-diazabicyclo[5,4,0]undecene, trimethylamine, diisopropylethylamine, and triethylamine, and a preferred example may be sodium hydroxide.

The amount of use of the base may be 1- to 10-fold the molar amount, and preferably 1- to 5-fold the molar amount, of the compound represented by the formula (XII). The molar ratio of the compound represented by the formula (XII) and the compound represented by the formula (XIII) may be such that compound represented by formula (XII)/compound represented by formula (XIII)=0.2 to 5. The reaction temperature may be from −10° C. to the reflux temperature, and is preferably from 0° C. to 80° C. The reaction time may be from 0.1 to 48 hours, and preferably from 0.1 to 12 hours. If the progress of reaction is delayed, a catalyst such as potassium iodide or sodium iodide may be added as necessary, in an amount of 0.1- to 1.5-fold the molar amount of the compound represented by formula (XII).

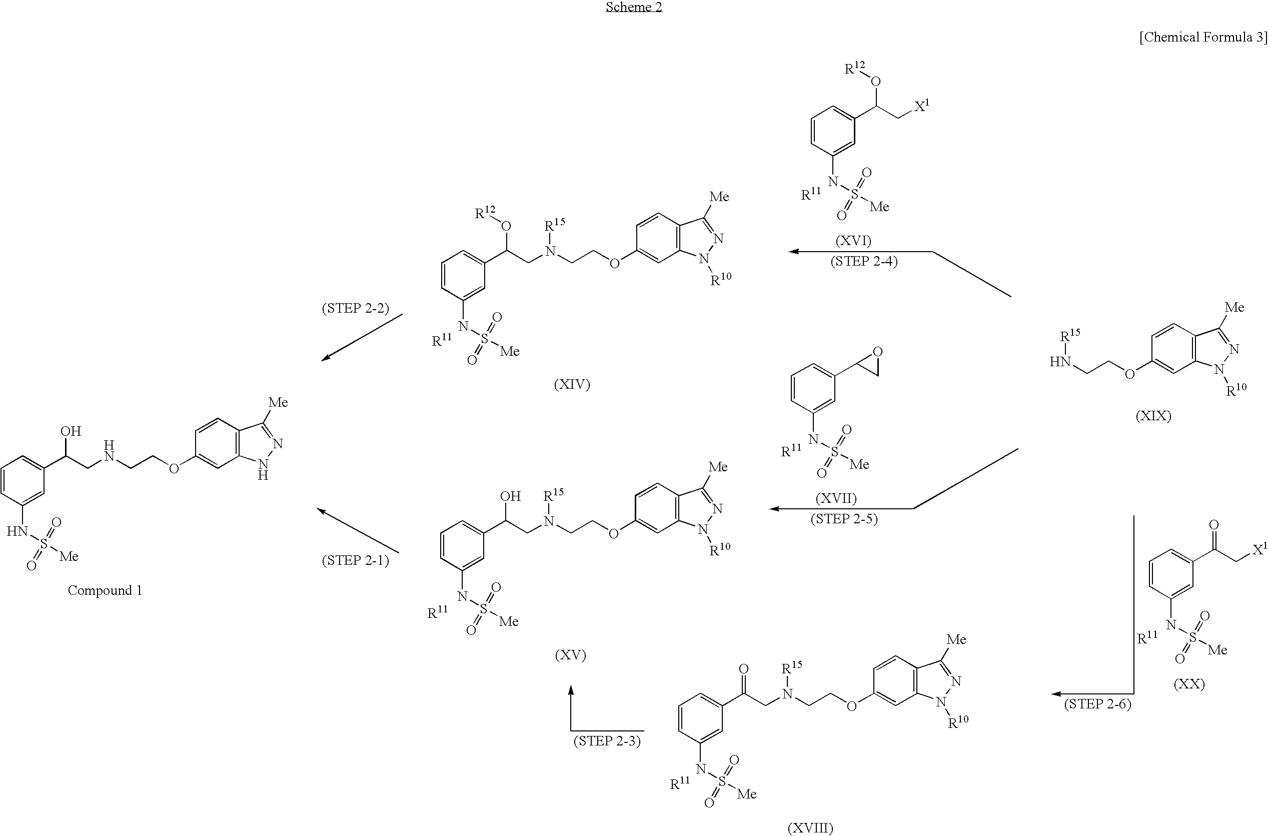

In the respective formulas in Scheme 2, R¹⁰ has the same meaning as defined above, and is preferably benzyl, tert-butoxycarbonyl or tetrahydropyranyl, and more preferably benzyl; R¹¹ has the same meaning as defined above, and is preferably benzyl; R¹² has the same meaning as defined above, and is preferably triethylsilyl or tart-butyldimethylsilyl; R¹⁵ is a hydrogen atom or a protective group for amino as described above, which is preferably benzyl; and X¹ is a leaving group, and examples thereof include chlorine atom, bromine atom, iodine atom, p-toluenesulfonyloxy and methanesulfonyloxy, preferably chlorine atom, bromine atom or iodine atom. Preferred combinations of R¹⁰, R¹¹, R¹² and R¹⁵ for the compound represented by the formula (XIV) include ones of: R¹⁰ (benzyl), R¹¹ (benzyl), R¹² (triethylsilyl) and R¹⁵ (benzyl); R¹⁰ (tert-butoxycarbonyl), R¹¹ (benzyl), R¹² (triethylsilyl) and R¹⁵ (benzyl); and R¹⁰ (tetrahydropyranyl), R¹¹ (benzyl), R¹² (triethylsilyl) and R¹⁵ (benzyl), more preferably the combination of R¹⁰ (benzyl), R¹¹ (benzyl), R¹² (triethylsilyl) and R¹⁵ (benzyl). Preferred combinations of R¹⁰, R¹¹ and R¹⁵ for the compound represented by the formula (XV) include ones of: R¹⁰ (benzyl), R¹¹ (benzyl) and R¹⁵ (benzyl); R¹⁰ (tert-butoxycarbonyl), R¹¹ (benzyl) and R¹⁵ (benzyl); and R¹⁰ (tetrahydropyranyl), R¹¹ (benzyl) and R¹⁵ (benzyl), more preferably the combination of R¹⁰ (benzyl), R¹¹ (benzyl) and R¹⁵ (benzyl). Preferred combinations of R¹⁰ and R¹⁵ for the compound represented by the formula (XIX) include ones of: R¹⁰ (benzyl) and R¹⁵ (benzyl); R¹⁰ (tert-butoxycarbonyl) and R¹⁵ (benzyl); and R¹⁰ (tetrahydropyranyl) and R¹⁵ (benzyl), more preferably the combination of R¹⁰ (benzyl) and R¹⁵ (benzyl).

Process 2-1 (STEP 2-1)

A compound represented by the formula (1) can be produced by subjecting the compound represented by the formula (XV) to a deprotection reaction according to a known method, for example, a method described in Protective Groups in Organic Synthesis, published by John Wiley and Sons (printed in 2007). As a suitable example, it is preferable to perform the deprotection reaction under acidic conditions as described above, or it is preferable to utilize the deprotection reaction based on hydrogenolysis as described above alone, or to utilize these deprotection reactions in combination. At any rate, it is desirable to select an appropriate deprotection reaction for each of the various protective groups present in the compound represented by the formula (XV). For example, in the case of a compound represented by the formula (XV) with R¹⁰ (benzyl), R¹¹ (benzyl) and R¹⁵ (benzyl), the deprotection reaction based on hydrogenolysis is preferred. The deprotection reaction based on hydrogenolysis may be a reaction carried out in an inert solvent, with a catalyst and hydrochloric acid added thereto, in the presence of hydrogen gas. A method of obtaining the compound represented by the formula (1) by subjecting the compound represented by the formula (XV) to a reaction in an inert solvent in the presence of a catalyst added thereto and hydrogen gas to thereby deprotect R¹¹ (benzyl) and R¹⁵ (benzyl), and then further adding hydrochloric acid to the reaction liquid to react therewith in the presence of hydrogen gas to deprotect R¹⁰ (benzyl), may be listed as a particularly preferred deprotection method.

In regard to the inert solvent, alcohols such as methanol or ethanol may be used singly, or a mixture of these may be used. Among them, ethanol is preferred. The catalyst is preferably a palladium-carbon powder.

The amount of use of the catalyst is preferably 2 to 40% by weight based on the compound represented by the formula (XV). The amount of use of hydrochloric acid is preferably 0.15- to 3-fold the molar amount of the compound represented by the formula (XV). The hydrogen gas used in the reaction is preferably under normal pressure or increased pressure. The reaction temperature may be from 20° C. to the reflux temperature, and is preferably from 30° C. to 60° C. The reaction time may be from 0.5 to 24 hours, and is preferably from 0.5 to 10 hours.

Process 2-2 (STEP 2-2)

The compound represented by the formula (1) can be produced by subjecting a compound represented by the formula (XIV) to a deprotection reaction according to a known method, for example, a method described in Protective Groups in Organic Synthesis, published by John Wiley and Sons (printed in 2007). As a suitable example, it is preferable to perform the deprotection reaction under acidic conditions as described above, or it is preferable to utilize the deprotection reaction based on hydrogenolysis as described above alone, or to utilize these deprotection reactions in combination. At any rate, it is desirable to select an appropriate deprotection reaction for each of the various protective groups present in the compound represented by the formula (XIV). For example, the deprotection reaction based on hydrogenolysis may be a method exemplified in the process 2-1 described above.

Process 2-3 (STEP 2-3)

This process can be carried out according to a method described in WO 2003/035620. That is, the compound represented by the formula (XV) can be obtained by allowing a compound represented by the formula (XVIII) to react with a reducing agent in an inert solvent.

Examples of such inert solvents include, e.g., alcohols such as methanol, ethanol and 2-propanol; tetrahydrofuran, dimethylformamide and dimethylsulfoxide. Examples of such reducing agents include sodium borohydride, sodium cyanoborohydride and borane.

Unless asymmetric reduction is especially carried out, the compound represented by the formula (XV), which is obtainable by this reduction reaction, can be obtained as a racemic mixture.

As the technique of obtaining an optically active form, there may be mentioned a technique of converting the racemic mixture into an addition salt with an optically active acid such as camphorsulfonic acid or mandelic acid, and then performing fractional crystallization to thereby separate an optically active form. A technique of separating using a commercially available column for optical resolution may also be mentioned. Alternatively, a technique of conducting asymmetric reduction may also be used. The asymmetric reduction reaction may be carried out by, for example, a method described in WO 2000/58287 (the disclosure of which is incorporated herein by reference), that is, a method of conducting asymmetric reduction together with a hydrogen supplying compound in the presence of a catalyst for asymmetric reduction, or the like.

Process 2-4 (STEP 2-4)

The compound represented by the formula (XIV) can be obtained by allowing a compound represented by the formula (XVI) to react with a compound represented by the formula (XIX) in an inert solvent, in the presence of a base added as necessary.

In regard to the inert solvent, N,N-dimethylformamide, N,N-dimethylacetamide, dimethylsulfoxide, acetonitrile or the like may be used singly, or a mixture of these may be used. Among them, N,N-dimethylformamide is preferred. Examples of the base include tertiary amines such as triethylamine, diisopropylethylamine, and 1,8-diazabicyclo[5,4,0]undecene; and alkali metal compounds such as potassium carbonate, sodium carbonate, cesium carbonate, and sodium hydrogen carbonate, and triethylamine or diisopropylethylamine is preferred.

The amount of use of the base may be, for example, 0 to 10-fold the molar amount, and preferably 0 to 5-fold the molar amount of the compound represented by the formula (XVI). The molar ratio of the compound represented by the formula (XVI) and the compound represented by the formula (XIX) is preferably such that compound represented by formula (XVI)/compound represented by formula (XIX)=0.2 to 5, and particularly preferably 0.5 to 2. The reaction temperature may be from −10° C. to the reflux temperature, and is preferably from 0° C. to 80° C. The reaction time may be from 0.1 to 48 hours, and is preferably from 2 to 20 hours.

If the progress of reaction is delayed, a catalyst such as potassium iodide or sodium iodide may be added as necessary, in an amount of 0.1- to 1.5-fold the molar amount of the compound represented by the formula (XVI).

Process 2-5 (STEP 2-5)

The compound represented by the formula (XV) can be obtained by allowing a compound represented by the formula (XVII) to react with the compound represented by the formula (XIX) in an inert solvent.

In regard to the inert solvent, an alcohol such as methanol, ethanol, 1-butanol, 2-butanol, or 2-propanol; N,N-dimethylformamide, N,N-dimethylacetamide, dimethylsulfoxide, acetonitrile or the like may be used singly, or a mixture of these may be used. Among them, 2-propanol is preferred.

The molar ratio of the compound represented by the formula (XVII) and the compound represented by the formula (XIX) is preferably such that compound represented by formula (XVII)/compound represented by formula (XIX)=0.2 to 5, and more preferably 0.75 to 1.5. The reaction temperature may be from −10° C. to the reflux temperature, and is preferably from 60° C. to the reflux temperature. The reaction time may be from 0.5 to 48 hours, and is preferably from 12 to 48 hours.

If necessary, a Lewis acid catalyst may be added.

Process 2-6 (STEP 2-6)

A compound represented by the formula (XVIII) can be obtained by allowing the compound represented by the formula (XIX) to react with a compound represented by the formula (XX) in an inert solvent, and if necessary, in the presence of a base added thereto. In regard to the inert solvent, N,N-dimethylformamide, N,N-dimethylacetamide, dimethylsulfoxide, acetonitrile or the like may be used singly, or a mixture of these may be used. Among them, N,N-dimethylformamide may be preferably mentioned as an example. Examples of the base include organic tertiary amines such as triethylamine, diisopropylethylamine and 1,8-diazabicyclo[5,4,0]undecene; and alkali metal compounds such as potassium carbonate, sodium carbonate, cesium carbonate, and sodium hydrogen carbonate, and triethylamine or diisopropylethylamine is preferred.

The amount of use of the base may be, for example, 0 to 10-fold the molar amount, and preferably 0 to 5-fold the molar amount of the compound represented by the formula (XX). The molar ratio of the compound represented by the formula (XIX) and the compound represented by the formula (XX) may be such that compound represented by formula (XIX)/compound represented by formula (XX)=0.2 to 5, and preferably 0.5 to 2. The reaction temperature may be from −10° C. to the reflux temperature, and is preferably from 0° C. to 80° C. The reaction time may be from 0.5 to 48 hours, and is preferably from 2 to 20 hours.

If the progress of reaction is delayed, a catalyst such as potassium iodide or sodium iodide may be added as necessary, in an amount of 0.1- to 1.5-fold the molar amount of the compound represented by the formula (XX).

The “compound 1” of the present invention thus obtainable, and the respective raw material compounds and intermediates can be isolated and purified according to conventional methods such as extraction, distillation, chromatography and crystallization.

Among the compounds used in the scheme 1 or 2, the compounds represented by the formulas (XI), (XII), (XIII), (XVI), (XVII), (XIX), and (XX) can be obtained by the methods as illustrated in scheme 3 to scheme 7. In the scheme 3 to scheme 7 illustrated below, the “STEP” is as defined above.

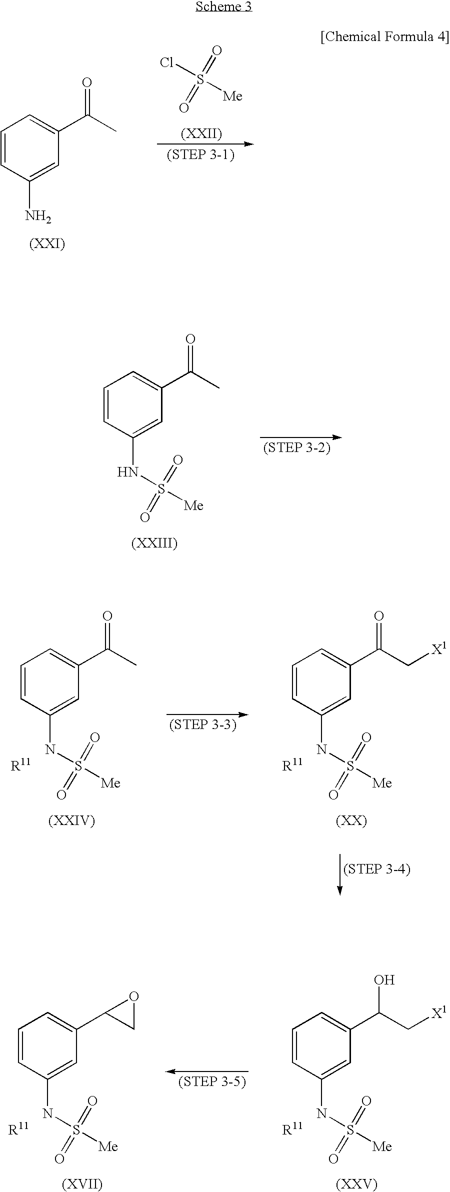

In the respective formulas in scheme 3, R¹¹ has the same meaning as defined above and is preferably benzyl; and X¹ has the same meaning as defined above and is preferably chlorine atom.

Process 3-1 (STEP 3-1)

A compound (XXIII) can be obtained by, for example, allowing 3-aminoacetophenone (XXI) that is commercially available from, e.g., Wako Pure Chemical Industries, Ltd. to react with methanesulfonyl chloride (XXII) that is commercially available from, e.g., Wako Pure Chemical Industries, Ltd., in an inert solvent in the presence of a base added thereto.

Examples of such inert solvents include hydrocarbon solvents such as toluene; halogenated hydrocarbons such as dichloromethane, chloroform, and 1,2-dichloroethane; and acetonitrile. Examples of such bases include organic bases such as triethylamine, N,N-diisopropylethylamine, and pyridine; and inorganic bases such as potassium carbonate, and sodium hydrogen carbonate.

The amount of use of the base may be 1- to 6-fold the molar amount, and preferably 1- to 3-fold the molar amount of 3-aminoacetophenone (XXI). The amount of use of methanesulfonyl chloride (XXII) may be usually 1- to 6-fold the molar amount, and preferably 1- to 3-fold the molar amount of 3-aminoacetophenone (XXI). The reaction temperature may be from −10° C. to 60° C., and is preferably from −10° C. to 30° C. The reaction time may be from 0.1 to 48 hours, and is preferably from 0.2 to 24 hours.

Process 3-2 (STEP 3-2)

A compound represented by the formula (XXIV) can be obtained by performing a protection reaction for the sulfonamide group of a compound (XXIII) according to a known method, for example, a method described in Protective Groups in Organic Synthesis, published by John Wiley and Sons (printed in 2007), or the like. As a suitable example, when R¹¹ is a benzyl group, a method of obtaining the compound represented by the formula (XXIV) by allowing the compound (XXIII) to react with a benzylating agent in an inert solvent in the presence of a base and a catalyst added thereto, may be used.

In regard to the inert solvent, ketones such as acetone, aprotic polar solvents such as N,N-dimethylformamide, or the like may be used singly, or a solvent mixture of these may be used. Examples of the benzylating agent include benzyl iodide, benzyl bromide, benzyl chloride, and the like, and benzyl chloride is preferred. Examples of the base include organic bases such as triethylamine, N,N-diisopropylethylamine and pyridine; and inorganic bases such as potassium carbonate and sodium hydrogen carbonate, and potassium carbonate is preferred. Examples of the catalyst include potassium iodide, sodium iodide and the like, and sodium iodide is preferred.

The amount of use of the base is preferably 1- to 5-fold the molar amount of the compound (XXIII). The amount of use of the catalyst is preferably 0.005- to 0.05-fold the molar amount of the compound (XXIII). The reaction temperature may be from 0° C. to the reflux temperature, and is preferably from 50° C. to 100° C. The reaction time is preferably from 1 to 24 hours.

Process 3-3 (STEP 3-3)

The compound represented by the formula (XX) can be obtained by allowing the compound represented by the formula (XXIV) to react in an inert solvent, with a halogenating agent added thereto, and with methanol further added as necessary.

Examples of the inert solvent include halogenated hydrocarbons such as dichloromethane, 1,2-dichloroethane and chloroform, and dichloromethane is preferred. Examples of the halogenating agent include chlorine gas, bromine gas, sulfuryl chloride, and the like, and sulfuryl chloride is preferred.

The amount of use of the halogenating agent is preferably 1- to 2-fold the molar amount of the compound represented by the formula (XXIV). The amount of use of methanol may be 0 to 5-fold the molar amount, and preferably 0.1- to 2-fold the molar amount of the compound represented by the formula (XXIV). The reaction temperature is preferably from −10° C. to 50° C. The reaction time, which includes the time for dropwise addition of the halogenating agent and methanol, is preferably from 1 to 10 hours.

Process 3-4 (STEP 3-4)

A compound represented by the formula (XXV) can be obtained by allowing the compound represented by the formula (XX) to react with a reducing agent in an organic solvent.

Examples of the organic solvent include alcohols such as methanol and ethanol; and ethers such as tetrahydrofuran. Examples of the reducing agent may be sodium borohydride and the like.

Unless an asymmetric reduction reaction is especially carried out, the compound represented by the formula (XXV), which is obtainable by this reduction reaction, can be obtained as a racemic mixture.

As the technique of obtaining an optically active form, a technique of conducting an asymmetric reduction reaction may be mentioned. The asymmetric reduction reaction can be carried out according to a method described in conventional literatures in chemistry, for example, a method described

In “Lectures on Experimental Chemistry, 4^{th} Edition” (edited by the Chemical Society of Japan, published by Maruzen Co., Ltd.), Vol. 26, pp. 23-68, or a method described in the reference documents cited therein. As a suitable example, there may be mentioned a method of obtaining the compound represented by the formula (XXV) by allowing the compound represented by the formula (XX) to react in an organic solvent, in the presence of a hydrogen source, with a catalyst added thereto.

Examples of the organic solvent include alcohols such as methanol, ethanol and 2-propanol; ethers such as tetrahydrofuran; halogenated hydrocarbons such as dichloromethane, 1,2-dichloroethane and chloroform; esters such as ethyl acetate; acetonitrile, and the like. These solvents may be used singly, or a mixture of these may be used. Among them, dichloromethane is preferred. Examples of the hydrogen source include hydrogen gas, a formic acid-triethylamine complex, and the like, and a formic acid-triethylamine complex is preferred. The catalyst may be an arene-chiral diamine-ruthenium (II) complex or the like, and examples thereof include [(s,s)-N-(p-toluenesulfonyl)-1,2-diphenylethylenediamine]-p-cymene-ruthenium complex, and [(s,s)-N-(p-toluenesulfonyl)-1,2-diphenylethylenediamine]-mesitylene-ruthenium complex.

The amount of use of the formic acid-triethylamine complex is preferably 1- to 10-fold the molar amount of the compound represented by the formula (XX), in terms of the mole number of formic acid. The ratio of the formic acid-triethylamine complex is preferably such that the amount of formic acid is 1- to 10-fold the molar amount of triethylamine. The amount of use of the catalyst may be such that the molar amount of compound represented by formula (XXV)/catalytic amount=S/C=10 to 10000, and the molar ratio is preferably such that S/C=100 to 1000. The reaction temperature may be from 0° C. to the reflux temperature, and is preferably from 20° C. to the reflux temperature. The reaction time, which includes the time for dropwise addition of the formic acid-triethylamine complex, may be from 0.1 to 24 hours, and is preferably from 0.5 to 12 hours.

Process 3-5 (STEP 3-5)

A compound represented by the formula (XVII) can be obtained by allowing the compound represented by the formula (XXV) to react in an inert solvent with a base added thereto.

In regard to the inert solvent, water, alcohols such as methanol or ethanol, N,N-dimethylformamide, tetrahydrofuran, 1,4-dioxane, acetone, 2-butanone, dimethylsulfoxide, acetonitrile or the like may be used singly, or a mixture of these may be used. Examples of the base include alkali metal compounds such as potassium carbonate, sodium carbonate, cesium carbonate, sodium hydrogen carbonate, potassium hydroxide, sodium hydroxide, sodium methoxide, a 28% sodium methoxide methanol solution, and potassium t-butoxide; and organic tertiary amines such as pyridine, 4-dimethylaminopyridine, 1,8-diazabicyclo[5,4,0]undecene, trimethylamine, and triethylamine.

The amount of use of the base is preferably 1- to 10-fold the molar amount of the compound represented by the formula (XXV). The reaction temperature may be from −40° C. to the reflux temperature, and is preferably from −10° C. to 50° C. The reaction time may be from 0.1 to 48 hours, and is preferably from 2 to 20 hours.

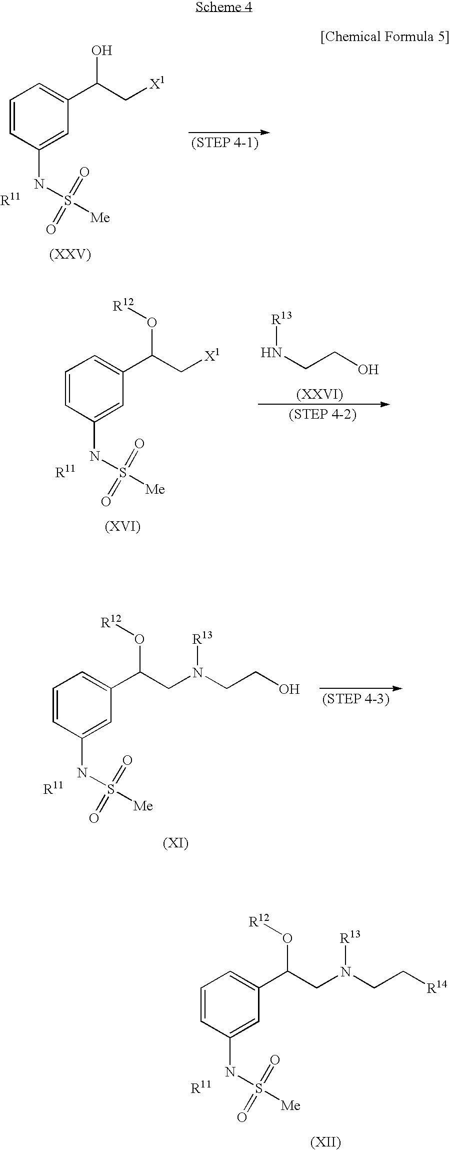

In the respective formulas in scheme 4, R¹¹ has the same meaning as defined above and is preferably benzyl or tert-butoxycarbonyl; R¹² has the same meaning as defined above and is preferably triethylsilyl or tert-butyldimethylsilyl; R¹³ has the same meaning as defined above and is preferably hydrogen atom, benzyl or tert-butoxycarbonyl; R¹⁴ has the same meaning as defined above and is preferably p-toluenesulfonyloxy, methanesulfonyloxy, or bromine atom; and X¹ has the same meaning as defined above and may be chlorine atom, bromine atom or iodine atom, with iodine atom being preferred. Preferred combinations of R¹¹, R¹² and R¹³ for the compound represented by the formula (XI) include ones of: R¹¹ (benzyl), R¹² (triethylsilyl) and R¹³ (benzyl); and R¹¹ (tert-butoxycarbonyl), R¹² (triethylsilyl) and R¹³ (tert-butoxycarbonyl).

Process 4-1 (STEP 4-1)

A compound represented by the formula (XVI) can be obtained by performing a protective reaction for the hydroxyl group of the compound represented by the formula (XXV), which is obtainable by the production method described in scheme 3 or the like, according to a known method, for example, a method described in Protective Groups in Organic Synthesis, published by John Wiley and Sons (printed in 2007), or the like. As a suitable example, there may be mentioned a method of obtaining the compound represented by the formula (XVI) by allowing the compound represented by the formula (XXV) to react with a silylating agent in an inert solvent in the presence of a base added thereto. Examples of such inert solvents include N,N′-dimethylformamide. Examples of such bases include imidazole. Examples of such silylating agents include triethylchlorosilane and tert-butyldimethylchlorosilane.

Process 4-2 (STEP 4-2)

This process can be carried out according to a method described in WO 2003/035620. That is, a compound represented by the formula (XI) can be obtained by allowing a compound represented by the formula (XVI) to react with a compound represented by the formula (XXVI) under the solventless conditions or in an inert solvent, in the presence of a base added as necessary.

In regard to the inert solvent, N,N-dimethylformamide, N,N-dimethylacetamide, dimethylsulfoxide, acetonitrile or the like may be used singly, or a mixture of these may be used. Among them, N,N-dimethylformamide is preferred. Examples of such bases include organic tertiary amines such as triethylamine, diisopropylethylamine, and 1,8-diazabicyclo[5,4,0]undecene; and alkali metal compounds such as potassium carbonate, sodium carbonate, cesium carbonate and sodium hydrogen carbonate, and triethylamine or diisopropylethylamine is preferred.

The amount of use of the base may be 0 to 10-fold the molar amount, and preferably 1- to 5-fold the molar amount of the compound represented by the formula (XVI). The amount of use of the compound represented by the formula (XXVI) may be 1- to 10-fold the molar amount, and preferably 1- to 5-fold the molar amount of the compound represented by the formula (XVI). The reaction temperature may be from −10° C. to the reflux temperature, and is preferably from 50° C. to the reflux temperature. The reaction time may be from 0.5 to 48 hours, and is preferably from 1 to 24 hours.

If the progress of reaction is delayed, a catalyst such as potassium iodide or sodium iodide may be added as necessary, in an amount of 0.1- to 1.5-fold the molar amount of the compound represented by the formula (XVI).

Process 4-3 (STEP 4-3)

A compound represented by the formula (XII) can be obtained by subjecting the compound represented by the formula (XI) to a method described in conventional literatures in chemistry, for example, a method described in “Lectures on Experimental Chemistry, 4^{th} Edition” (edited by the Chemical Society of Japan, published by Maruzen Co., Ltd.), Vol. 19, pp. 438-446, or a method described in the reference documents cited therein. As a suitable example, there may be mentioned a method of obtaining the compound represented by the formula (XII) by allowing the compound represented by the formula (XI) to react with a halogenating reagent and a phosphine added thereto, in an inert solvent.

In regard to the inert solvent, halogenated hydrocarbons such as dichloromethane or chloroform; ethers such as tetrahydrofuran; hydrocarbons such as benzene or toluene; or the like may be used singly, or a mixture of these may be used. Among them, dichloromethane is preferred. Examples of such halogenating reagents include carbon tetrachloride, N-chlorosuccinimide, N-bromosuccinimide, carbon tetrachloride, N-iodosuccinimide and the like, and N-bromosuccinimide is preferred. Examples of such phosphines include triphenylphosphine, n-butylphosphine and the like, and triphenylphosphine is preferred.

The amount of use of the halogenating reagent is preferably 1- to 10-fold the molar amount of the compound represented by the formula (XI). The amount of use of the phosphine is preferably 1- to 10-fold the molar amount of the compound represented by the formula (XI). The reaction temperature may be from −10° C. to the reflux temperature, and is preferably from −10° C. to 40° C. The reaction time may be from 0.1 to 24 hours, and is preferably from 0.5 to 12 hours.

Furthermore, the compound represented by the formula (XII) can be obtained by allowing the compound represented by the formula (XI) to react with a halogenating reagent in an inert solvent, in the presence of a base added as necessary.

In regard to the inert solvent, halogenated hydrocarbons such as dichloromethane or chloroform; ethers such as tetrahydrofuran; hydrocarbons such as benzene or toluene; or the like may be used singly, or a mixture of these may be used. Examples of the halogenating reagent include thionyl chloride, thionyl bromide, and the like. Examples of the base include organic tertiary amines such as triethylamine, diisopropylethylamine, and 1,8-diazabicyclo[5,4,0]undecene; and the like.

The amount of use of the halogenating reagent is preferably 1- to 10-fold the molar amount of the compound represented by the formula (XI). The amount of use of the base may be 0 to 10-fold the molar amount, and preferably 1- to 10-fold the molar amount, of the compound represented by the formula (XI). The reaction temperature may be from −10° C. to the reflux temperature, and is preferably from −10° C. to 40° C. The reaction time may be from 0.1 to 24 hours, and is preferably from 0.5 to 12 hours.

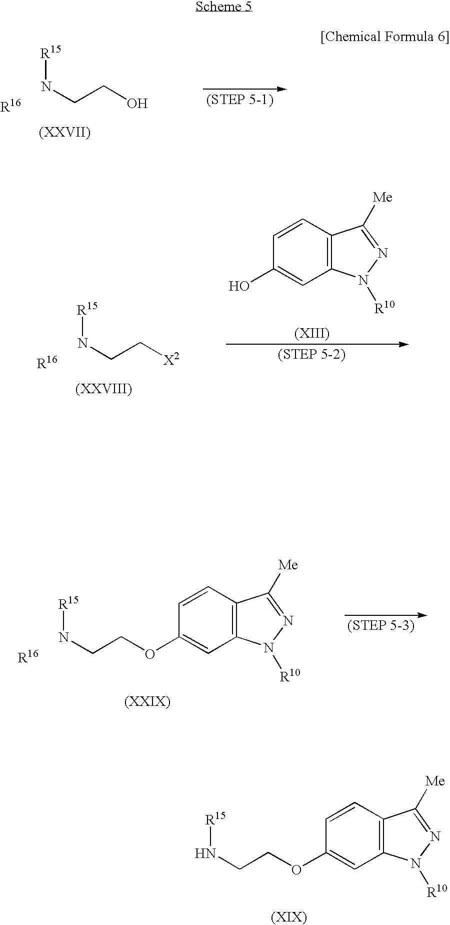

In the respective formulas in scheme 5, R¹⁰ has the same meaning as defined above and is preferably benzyl, tert-butoxycarbonyl or tetrahydropyranyl, more preferably benzyl; R¹⁵ has the same meaning as defined above and is preferably benzyl; and R¹⁶ is hydrogen atom or a protective group for amino, and if R¹⁶ is a protective group for amino, R¹⁶ is preferably a group identical to R¹⁵ or a group that can be selectively deprotected in preference to R¹⁵. According to another embodiment, a combination in which R¹⁵ is a group that can be selectively deprotected in preference to R¹⁶, is preferred. X² represents a leaving group and may be chlorine atom, bromine atom, iodine atom, p-toluenesulfonyloxy or methanesulfonyloxy. A preferred combination of R¹⁵ and R¹⁶ for the compound represented by the formula (XXVII) is R¹⁶ (benzyl) and R¹⁶ (benzyl). Preferred combinations of R¹⁰, R¹⁵ and R¹⁶ for the compound represented by the formula (XXIX) include ones of: R¹⁰ (benzyl), R¹⁵ (benzyl) and R¹⁶ (benzyl); R¹⁰ (tert-butoxycarbonyl), R¹⁵ (benzyl) and R¹⁶ (benzyl); and R¹⁰ (tetrahydropyranyl), R¹⁵ (benzyl) and R¹⁶ (benzyl), more preferably the combination of R¹⁰ (benzyl), R¹⁵ (benzyl) and R¹⁶ (benzyl). A preferred combination of R¹⁰ and R¹⁵ for the compound represented by the formula (XIX) is one of R¹⁰ (benzyl) and R¹⁵ (benzyl).

For example, a compound represented by the formula (XXVII) with R¹⁵ (benzyl) and R¹⁶ (benzyl); a compound represented by the same formula with R¹⁵ (benzyl) and R¹⁶ (hydrogen atom); a compound represented by the same formula with R¹⁵ (hydrogen atom) and R¹⁶ (hydrogen atom) are available from Tokyo Chemical Industry Co., Ltd. or the like.

Process 5-1 (STEP 5-1)

A compound represented by the formula (XXVIII) can be obtained by allowing the compound represented by the formula (XXVII) to react in an inert solvent, with a base and a sulfonylating reagent added thereto.

In regard to the inert solvent, halogenated hydrocarbons such as dichloromethane or chloroform; or ethers such as tetrahydrofuran may be used singly, or a mixture of these may be used. Examples of the base include organic tertiary amines such as pyridine, triethylamine, diisopropylethylamine, and 1,8-diazabicyclo[5,4,0]undecene; and alkali metal compounds such as potassium carbonate, sodium carbonate, cesium carbonate and sodium hydrogen carbonate. Examples of the sulfonylating reagent include p-toluenesulfonyl chloride, methanesulfonyl chloride, and the like.

The amount of use of the sulfonylating reagent may be 1- to 10-fold the molar amount, and preferably 1- to 2-fold the molar amount, of the compound represented by the formula (XXVII). The amount of use of the base may be 1- to 10-fold the molar amount, and preferably 1- to 2-fold the molar amount, of the compound represented by the formula (XXVII). The reaction temperature may be from −20° C. to the reflux temperature, and is preferably from −10° C. to 50° C. The reaction time may be typically from 0.1 to 24 hours, and the time including the time for dropwise addition of the reagent is preferably from 1 to 10 hours.

Furthermore, the compound represented by the formula (XXVIII) may also be obtained by subjecting the compound represented by the formula (XXVII) to a method described in conventional literatures in chemistry, for example, a method described in “Lectures on Experimental Chemistry, 4^{th} Edition” (edited by the Chemical Society of Japan, published by Maruzen Co., Ltd.), Vol. 19, pp. 438-446, or a method described in the reference documents cited therein. As a suitable example, there may be mentioned a method of obtaining the compound represented by the formula (XXVIII) by allowing the compound represented by the formula (XXVII) to react in an inert solvent, with a halogenating reagent and a phosphine added thereto.

In regard to the inert solvent, halogenated hydrocarbons such as dichoromethane or chloroform; ethers such as tetrahydrofuran; hydrocarbons such as benzene or toluene; or the like may be used singly, or a mixture of these may be used. Examples of the halogenating reagent include carbon tetrachloride, N-chlorosuccinimide, N-bromosuccinimide, carbon tetrabromide, N-iodosuccinimide, and the like. Examples of the phosphine include triphenylphosphine, n-butylphosphine, and the like, and triphenylphosphine is preferred.

The amount of use of the halogenating reagent is preferably 1- to 10-fold the molar amount of the compound represented by the formula (XXVII). The amount of use of the phosphine is preferably 1- to 10-fold the molar amount of the compound represented by the formula (XXVII). The reaction temperature may be from −10° C. to the reflux temperature, and is preferably from −10° C. to 40° C. The reaction time may be from 0.1 to 24 hours, and is preferably from 0.5 to 12 hours.

In still another method, the compound represented by the formula (XXVIII) can be obtained by allowing the compound represented by the formula (XXVII) to react with a halogenating reagent in an inert solvent, in the presence of a base if necessary.

In regard to the inert solvent, halogenated hydrocarbons such as dichloromethane or chloroform; ethers such as tetrahydrofuran; hydrocarbons such as benzene or toluene; or the like may be used singly, or a mixture of these may be used.

Examples of the halogenating reagent include thionyl chloride, thionyl bromide, phosphorus tribromide, and the like. Examples of the base include organic tertiary amines such as pyridine, 4-dimethylaminopyridine, triethylamine, diisopropylethylamine, and 1,8-diazabicyclo[5,4,0]undecene; and the like.

The amount of use of the halogenating reagent is preferably 1- to 10-fold the molar amount of the compound represented by the formula (XXVII). The amount of use of the base may be 0 to 10-fold the molar amount, and preferably 1- to 10-fold the molar amount, of the compound represented by the formula (XXVII). The reaction temperature may be from −10° C. to the reflux temperature, and is preferably from −10° C. to 40° C. The reaction time may be from 0.1 to 24 hours, and is preferably from 0.5 to 12 hours.

Process 5-2 (STEP 5-2)

A compound represented by the formula (XXIX) can be obtained by allowing the compound represented by the formula (XIII) to react with the compound represented by the formula (XXVIII) in an inert solvent, in the presence of a base added thereto.

In regard to the inert solvent, tetrahydrofuran, N,N-dimethylformamide, N,N-dimethylacetamide, dimethyl sulfoxide, acetonitrile or the like may be used singly, or a mixture of these may be used. Examples of the base include alkali metal compounds such as potassium carbonate, sodium carbonate, cesium carbonate, sodium hydrogen carbonate, potassium hydroxide, sodium hydroxide, sodium methoxide, a 28% sodium methoxide methanol solution, and potassium t-butoxide; and organic tertiary amines such as pyridine, 4-dimethylaminopyridine, 1,8-diazabicyclo[5,4,0]undecene, trimethylamine, and triethylamine.

The amount of use of the base may be 1- to 10-fold the molar amount, and preferably 1- to 5-fold the molar amount, of the compound represented by the formula (XIII). The amount of use of the compound represented by the formula (XXVIII) may be 1- to 10-fold the molar amount, and preferably 1- to 3-fold the molar amount, of the compound represented by the formula (XIII). The reaction temperature may be from −20° C. to the reflux temperature, and is preferably from 0° C. to 60° C. The reaction time may be from 0.1 to 48 hours, and preferably, the time including the time for dropwise addition of the reagent may be 2 to 24 hours.

If the progress of reaction is delayed, a catalyst such as potassium iodide or sodium iodide may be added as necessary, in an amount of 0.1- to 1.5-fold the molar amount of the compound represented by the formula (XXVIII).

Process 5-3 (STEP 5-3)

When removal of the protective group of the compound represented by the formula (XXIX) is needed, a deprotection reaction for R¹⁶ may be selectively carried out in preference to R¹⁰ and R¹⁵, according to a known method, for example, a method described in Protective Groups in Organic Synthesis, published by John Wiley and Sons (printed in 2007), or the like. According to another embodiment, a deprotection reaction for R¹⁵ may be selectively carried out in preference to R¹⁰ and R¹⁶. Pox example, if R¹⁵ and R¹⁶ in the formula (XXIX) are all benzyl groups, conditions in which only one of the benzyl groups of R¹⁵ and R¹⁶ is selectively deprotected, may be mentioned. A method involving such conditions may be a method of obtaining the compound represented by the formula (XIX) by a reaction in an inert solvent in the presence of hydrogen gas under normal pressure or increased pressure, while controlling the reaction by adding a catalyst and hydrochloric acid.

Examples of the inert solvent include alcohols such as methanol or ethanol, and ethanol is preferred. The catalyst is preferably a palladium-carbon powder.

The amount of use of the catalyst may be 1 to 40% by weight, and preferably 5 to 40% by weight, based on the compound represented by the formula (XXIX). The amount of use of hydrochloric acid may be 0.05- to 3-fold the molar amount, and preferably 0.1- to 1-fold the molar amount, of the compound represented by the formula (XXIX). The reaction temperature may be from 0° C. to 60° C., and is preferably from 0° C. to 40° C. The reaction time may be from 0.1 to 24 hours, and is preferably from 0.1 to 12 hours.

The compound represented by the formula (XXIX) can also be obtained according to the method described in scheme 6.

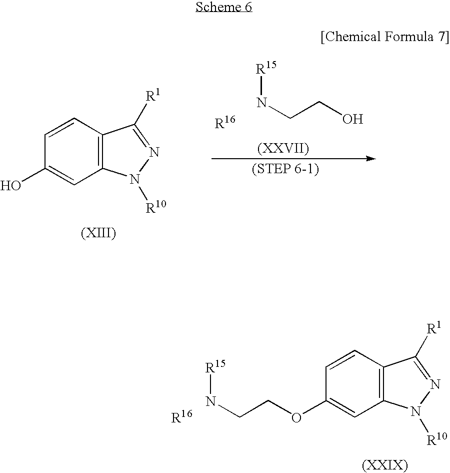

In the respective formulas in scheme 6, R¹⁰, R¹⁵ and R¹⁶ have the same meanings as defined above.

Process 6-1 (STEP 6-1)

The compound represented by the formula (XXIX) can be obtained by allowing the compound represented by the formula (XIII) to react with the compound represented by the formula (XXVII) in an inert solvent, in the presence of a phosphine and an azo compound added thereto.

Examples of the inert solvent include ethers such as diethyl ether, tetrahydrofuran, and dimethoxyethane; halogenated solvents such as methylene chloride; and benzene analogs such as benzene, toluene and xylene, and toluene or tetrahydrofuran is preferred. Examples of the phosphine include triphenylphosphine or tributylphosphine, and triphenylphosphine is preferred. Examples of the azo compound include diethyl azodicarboxylate, diisopropyl azodicarboxylate, N,N,N′,N′-tetramethylazodicarboxamide, 1,1′-(azodicarbonyl)dipiperidine, N,N,N′,N′-tetraisopropylcarboxamide, and the like, and N,N,N′,N′-tetramethylazodicarboxamide is preferred.

The amount of use of the phosphine may be 1- to 10-fold the molar amount, and preferably 1- to 5-fold the molar amount, of the compound represented by the formula (XIII). The amount of use of the azo compound may be 1- to 10-fold the molar amount, and 1- to 5-fold the molar amount, of the compound represented by the formula (XIII). The amount of use of the compound represented by the formula (XXVII) may be 1- to 10-fold the molar amount, and preferably 1- to 5-fold the molar amount, of the compound represented by the formula (XIII). The reaction temperature may be usually from −20° C. to the reflux temperature, and is preferably from 0° C. to 30° C. The reaction time may be from 1 to 48 hours, and is preferably from 3 to 24 hours.

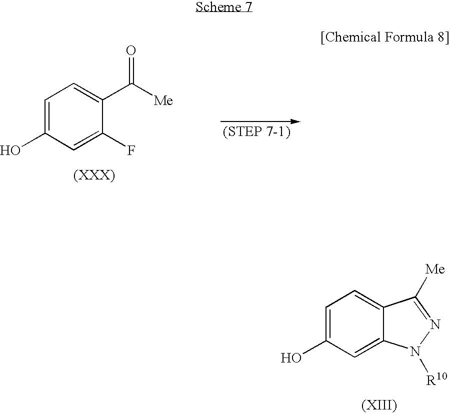

In the respective formulas in scheme 7, R¹⁰ has the same meaning as defined above, and is preferably a benzyl group, a tert-butoxycarbonyl group, or a tetrahydropyranyl group, and more preferably a benzyl group.

Process 7-1 (STEP 7-1)

The compound (XIII) can be obtained by allowing a compound (XXX) which is available from Tokyo Chemical Industry Co., Ltd. or the like, to react in an inert solvent, with a hydrazine added thereto, and if necessary, in the presence of a base added thereto.

In regard to the inert solvent, alcohols such as methanol, ethanol, 1-butanol or 2-butanol; ethers such as tetrahydrofuran or dimethoxyethane; benzene analogs such as benzene, toluene or xylene; or the like may be used singly, or a mixture of these may be used. Among them, xylene is preferred. Examples of the hydrazine include benzylhydrazine, benzylhydrazine monohydrochloride, benzylhydrazine dihydrochloride, hydrazine monohydrochloride, hydrazine dihydrochloride, hydrazine monohydrate, and hydrazine hydrate, and benzylhydrazine monohydrochloride is preferred. Examples of the base include alkali metal compounds such as sodium acetate, potassium carbonate, sodium carbonate, cesium carbonate and sodium hydrogen carbonate, and the like, and sodium acetate is preferred.

The amount of use of the hydrazine may be 1- to 5-fold the molar amount, and preferably 1- to 3-fold the molar amount, of the compound represented by the formula (XXX). The amount of use of the base may be 0 to 10-fold the molar amount, and preferably 1- to 5-fold the molar amount, of the compound represented by the formula (XXX). The reaction temperature may be from 0° C. to the reflux temperature, and is preferably from 50° C. to the reflux temperature. The reaction time may be from 0.1 to 48 hours, and is preferably from 3 to 24 hours.

If the progress of reaction is delayed, the reaction can be carried out under increased pressure in the reaction system by sealing the reaction vessel. In this case, it is possible to carry out the reaction at a temperature above the reflux temperature of the solvent, and the reaction temperature may be from the reflux temperature to 250° C., and preferably from the reflux temperature to 200° C.

In order to estimate the type of the acid that forms a salt with the “compound 1” and the number of molecules of the added acid, the number of molecules of the added acid per molecule of the “compound 1” may be calculated by ion exchange chromatography. For example, use is made of a method in which the added acid is dissociated by ion exchange using a DIONEX IonPac AS14 column for ion exchange chromatography (manufactured by Dionex Corp.) having an internal diameter of 4 mm and a length of 25 cm and the like, and the acid is quantified by comparing the peak area with the peak area of a known standard ion solution using an electrical conductivity detector, to thereby calculate the number of molecules of the added acid per molecule of the “compound 1.”

Furthermore, the type of the acid that forms a salt with the “compound 1” and the number of molecules of the added acid can also be estimated according to a technique such as quantification of the amount of element by elemental analysis. If a single crystal is obtained, the type of the acid that forms a salt with the “compound 1” and the number of molecules of the acid can also be estimated by X-ray structural analysis.

It is well known to those having ordinary skill in the art that slight errors may occur in the measurement of the number of molecules of the added acid measured by ion chromatography, due to various factors. A measurement error that is tolerated in the number of molecules of the added acid per molecule of the “compound 1,” is usually ±0.2 molecules, and preferably ±0.1 molecules.

As a test for identifying the “dihydrochloride salt”, the powder X-ray diffraction method can be used. Furthermore, it is also acceptable to measure the infrared absorption spectrum and use it for the identification. More specifically, there may be mentioned a method of measuring an infrared absorption spectrum using a powder of the “dihydrochloride salt”, and for example, the potassium bromide tablet method described in the section of “Infrared Absorption Spectrometry” under General Tests, Processes and Apparatus in the Japanese Pharmacopoeia, or the like can be selected.

As the technique of evaluating the purity of the “dihydrochloride salt”, an area percentage method associated with the HPLC method is convenient. As the technique of evaluating the moisture content of the “dihydrochloride salt”, the volumetric titration method and the coulometric titration method described in the section of “Water Determination” under General Tests, Processes and Apparatus in the Japanese Pharmacopoeia, or a dry weight reduction measurement method or the like can be used. If the weight of the sample is small, it is preferable to select the coulometric titration method.

When it is needed to measure the amount of presence of the “dihydrochloride salt” contained in the preparation, it is usually convenient and preferable to use HPLC. For example, a calibration curve for the “dihydrochloride salt” can be produced by the HPLC method using a standard product of the “dihydrochloride salt” of known chemical purity, and the amount of presence of the “dihydrochloride salt” in the sample can be quantified on the basis of this calibration curve.

The optical system used in the measurement of the powder X-ray diffraction spectrum may be, for example, a generally used a focusing beam optical system or a parallel beam optical system. The optical system used is not particularly limited, while if it is intended to secure the resolution or intensity, it is preferable to make measurements using a focusing beam optical system. Furthermore, when it is intended to suppress orientation, which is a phenomenon of crystals aligning in a constant direction due to the crystal shape (needle shape, plate shape, or the like), it is preferable to make measurements using a parallel beam optical system. Examples of the measuring apparatus for the focusing beam optical system include XRD-6000 (manufactured by Shimadzu Corp.), MultiFlex (manufactured by Rigaku Corp.), and the like. Examples of the measuring apparatus for the parallel beam optical system include XRD-7700 (manufactured by Shimadzu Corp.), RINT 2200 Ultima+/PC (manufactured by Rigaku Corp.), and the like.

It is well known to those having ordinary skill in the art that there may occur slight errors in the measurement of the 2θ value in the powder X-ray diffraction spectrum due to various factors. Usually, a measurement error of about ±0.3°, typically ±0.2°, and preferably ±0.1°, is tolerated. Therefore, it will be understood by those having ordinary skill in the art that the value of 20 expressed additionally with the term “approximately” may include a tolerable measurement error.

It is well known to those having ordinary skill in the art that a measurement value obtainable by differential scanning calorimetric analysis is a value intrinsic to the crystal of the object of measurement, but it is also known to those having ordinary skill in the art that in addition to the measurement error against the actual measurement, there is a possibility of fluctuation in the melting point occurring under certain circumstances due to causes such as the incorporation of a tolerable amount of impurities, and the like. Accordingly, a person having skill in the art can understand that the actually measured value of the peak temperature in the differential scanning calorimetric analysis as described herein may fluctuate under certain circumstances, and can also understand that the range of fluctuation is, for example, about ±5° C., typically about ±3° C., and preferably about ±2° C. The measuring apparatus used in the differential scanning calorimetric analysis may be, for example, PYRIS Diamond DSC (manufactured by PerkinElmer, Inc.), DSC3200 (manufactured by Bruker AXS, Inc.), or the like.

Slight errors in measurement may also be tolerated with regard to the wavenumber of the infrared absorption spectrum, and it will be readily understood by those having ordinary skill in the art that it is tolerated that the values described herein include such measurement errors. For example, according to the 4^{th} edition of the European Pharmacopoeia, in a comparison of the measured spectrum with a reference spectrum in an identification test based On the infrared absorption spectrum, an agreement within ±0.5% of the wavenumber scale is acceptable. Although the present specification is not intended to be bound by this criterion of judgment, for example, as one criterion for judgment, a measurement error of about ±0.8%, preferably about ±0.5%, and particularly preferably about ±0.2%, with regard to the wavenumber scale is tolerated.

The thermal stability of the “dihydrochloride salt” can be evaluated by, for example, sealing a sample in a glass vial or the like, storing the sample for a certain time period in a dark place, for example, under a harsh temperature condition of about 40° C. to 80° C., and then measuring the properties, purity, moisture content and the like of the compound 1 or a substance in the form of a salt thereof. Particularly, the change in purity before and after the storage serves as an important index for thermal stability. For example, it is preferable to carry out the evaluation under a storage condition of 60° C.

The hygroscopic property of the “dihydrochloride salt” can be evaluated by, for example, placing a sample on a weighing pan made of glass, storing the sample for a certain time period in an open state in a dark place, for example, under humidified conditions at a temperature of 25° C. to 40° C. and a humidity of about 75% to 941, and then measuring the properties, purity, moisture content and the like of the compound 1 or a substance in the form of a salt thereof. Particularly, the increment of the moisture content before and after the storage serves as an important index for the hygroscopic property. It is preferable to evaluate, for example, under storage conditions of 25° C./84% RH.

In regard to a composition containing the “dihydrochloride salt”, when attention is paid to (R)—N-[3-[2-[2-(3-methylindazol-6-yloxy)ethylamino]-1-hydroxyethyl]phenyl]methanesulfonamide and a salt thereof, and solvates of the compound and the salt, and the total mass of these compounds is considered as 100%, the proportion by mass of the “dihydrochloride salt” occupying in the total mass can be set to about 50%, about 60%, about 70% or about 80% or more, and any value with which the effects of the “dihydrochloride salt” of the present invention are verified may be taken. For example, the proportion by mass is preferably about 90% or more, more preferably about 95% or more, even more preferably 96% or more, still more preferably 97% or more, particularly preferably 98% or more, and particularly highly preferably near 100%.

A medicine containing the dihydrochloride salt of the present invention or a crystal thereof as an active ingredient, is useful for the prevention and/or treatment of diabetes mellitus, obesity, hyperlipidemia, overactive bladder, urinary incontinence and the like.

That is, the dihydrochloride salt of the present invention is not recognized as toxic and is useful as a medicine. For example, since the dihydrochloride salt has β3 adrenergic receptor agonist activity, the salt can be used as a medicine used in the treatment and prevention of β3 adrenergic receptor-associated diseases. The β3 adrenergic receptor-associated diseases is a collective term for the diseases which can be improved by the agonist activity mediated by the subject receptor, and examples thereof include overactive bladder, urinary incontinence, interstitial cystitis, diabetes mellitus, obesity, hyperlipidemia, fatty liver, digestive diseases (preferably, abnormal movement or ulcers in the digestive system), depression, diseases caused by gallstones or hypermotility of the biliary tract, and the like. Particularly, it is more preferable to use the medicine of the present invention for the treatment and/or prevention of overactive bladder or urinary incontinence, and it is particularly preferable to use the medicine of the present invention for the treatment of overactive bladder. According to another embodiment, it is particularly preferable to use the medicine of the present invention for the treatment of urinary incontinence. According to the International Continence Society (ICS), overactive bladder is defined as “urgency of urination as a main symptom, with or without urgency incontinence, usually with frequency and nocturia.” Furthermore, according to the International Continence Society, urinary incontinence is defined as “a condition where involuntary loss of urine is a social or hygienic problem and is objectively demonstrable.”

The fact that the dihydrochloride salt of the present invention is useful for the treatment and/or prevention of the diseases described above, for example, overactive bladder, urinary incontinence, interstitial cystitis and the like, can be confirmed by performing a test with reference to, for example, British Journal of Pharmacology, vol. 122, pp. 1720-1724 (1997), and observing the smooth muscle relaxing action of the test compound in the urinary bladder extracted from a common marmoset.

In a specific example, a common marmoset (CLEA Japan, Inc.) is killed by exsanguination, and then is subjected to laparotomy to extract the urinary bladder. A smooth muscle specimen is produced from the extracted bladder, and then the specimen is suspended in an organ bath filled with 10 mL Krebs-Henseleit fluid aerated with a mixed gas of 95% O₂ and 5% CO₂. The specimen is placed under a resting tension of 1 g, and is stabilized for 30 minutes or longer. After the resting tension of the specimen has been stabilized, KCl at a final concentration of 40 mmol/L is repeatedly added to the organ bath, and it is confirmed that contraction against KCl becomes almost constant. After the generated tension is stabilized by contracting the specimen using KCl at a final concentration of 40 mmol/L, the test compound is cumulatively added (at an interval of 20 minutes) at a ratio of 10 times, and the relaxation response is observed. The final concentration is set at 10⁻⁹, 10⁻⁸, 10⁻⁷, 10⁻⁸, 10⁻⁵ and 10⁻⁴ mol/L. When the relaxation response at the maximum concentration of the test compound is completed, papaverine at a final concentration of 10⁻⁴ mol/L is added, and the maximum relaxation response of each specimen is determined. With this relaxation response taken as 100%, the ratios of relaxation (%) obtainable at test compound concentrations of 10⁻⁵ mol/L and 10⁻⁴ mol/L are calculated, and thereby the bladder smooth muscle relaxing action can be evaluated.

Likewise, the fact that the dihydrochloride salt of the present invention is useful for the treatment and/or prevention of the diseases described above, for example, overactive bladder, urinary incontinence, interstitial cystitis and the like, can be confirmed by performing a test with reference to, for example, The Journal of Urology, vol. 170, pp. 649-653 (2003), and observing the smooth muscle relaxing action of the test compound in the urinary bladder extracted from human being.

That is, a smooth muscle specimen obtained from a human-extracted urinary bladder is suspended in an organ bath filled with 10 mL Krebs-Henseleit fluid aerated with a mixed gas of 95% O₂ and 5% CO₂. The specimen is placed under a resting tension of 1 g, and is stabilized for 30 minutes or longer. After the resting tension of the specimen has been stabilized, carbachol at a final concentration of 0.1 μmol/L is repeatedly added to the organ bath, and it is confirmed that contraction against carbachol becomes almost constant. After the generated tension is stabilized by contracting the specimen using carbachol at a final concentration of 0.1 μmol/L, the test compound is cumulatively added (at an interval of 10 minutes) at a ratio of 10 times, and the relaxation response is observed. The final concentration is set at 10⁻⁹, 10⁻⁸, 10⁻⁷, 10⁻⁶, 10⁻⁵ and 10⁻⁴ mol/L. When the relaxation response at the maximum concentration of the test compound is completed, papaverine at a final concentration of 10⁻⁴ mol/L is added, and the maximum relaxation response of each specimen is determined. With this relaxation response taken as 100%, the ratios of relaxation (%) obtainable at test compound concentrations of 10⁻⁵ mol/L and 10⁻⁴ mol/L are calculated.

It is needless to say, the tests described above are only non-limiting examples of the method of checking the bladder smooth muscle relaxing action, and the bladder smooth muscle relaxing action can be confirmed according to any method that is well known to those having ordinary skill in the art.

When a medicine containing the dihydrochloride salt of the present invention as an active ingredient is administered to human being, the medicine can be orally administered in the form of a tablet, a powder, a granule, a capsule, a sugar-coated tablet, a liquid, a syrup or the like, or can be parenterally administered in the form of an injectable preparation, an infusion preparation, a suppository, a transdermal or absorptive preparation, or the like. Furthermore, inhalation in the form of a sprayable preparation such as an aerosol or a dry powder may also be mentioned as a preferred form of administration. It is also a preferred embodiment that the medicine containing the crystals of the dihydrochloride salt of the present invention as an active ingredient is a medicine in a solid form.

The period of administration for the medicine of the present invention is not particularly limited, and in the case of administering the medicine for therapeutic purposes, a time period in which the clinical symptoms of each of the diseases are considered to be expressed, may be selected in principle as the period of administration. Typically, it is general to continue administration for several weeks to a whole year, though administration can be further continued in accordance with the pathological condition, and continuous administration after the recovery of clinical symptoms is also allowed. Even if the clinical symptoms are no longer expressed, the medicine can also be administered for the prophylactic purposes at a clinician's discretion. The dose of the medicine of the present invention is not particularly limited, for example, generally 0.01 to 2000 mg of the active ingredient can be administered once or in several divided portions a day for an adult. The frequency of administration can range from once monthly to daily administration, and the frequency preferably ranges from once weekly to three times weekly or five times weekly, or daily administration. The daily dose, period of administration and frequency of administration can all be appropriately increased or decreased depending on the age, body weight and the degree of physical healthiness of the patient, and the disease to be treated, the severity of the disease or the like.

It will be appreciated that the medicine in accordance with an embodiment of the present invention can be administered together with a prophylactic or therapeutic drug for various abnormalities or diseases, in addition to the purpose of prevention and/or treatment of the medicine in accordance with an embodiment of the present invention.

### EXAMPLES

The present invention will now be more specifically explained by way of Reference Examples, Examples and Test Examples, but these examples are not intended to limit the scope of the present invention.

### Reference Example 1a

### Synthesis of (R)—N-[3-[2-[2-(3-methylindazol-6-yloxy)ethylamino]-1-hydroxyethyl]phenyl]methanesulfonamide, “compound 1”

To 57.05 g (84.54 mmol) of (R)—N-benzyl-N-[3-[2-N′-benzyl-2-(1-benzyl-3-methylindazol-6-yloxy)ethylamino]-1-hydroxyethyl]phenyl]methanesulfonamide that can be produced according to a method described in WO 2003/035620, 817.5 ml of ethanol, 14.5 ml of hydrochloric acid, and 11.4 mg of 10% palladium-carbon (water content 55%, PE type manufactured by N.E. Chemcat Corp., Lot No. 217-015551) were added, and the system was purged with hydrogen. This solution was warmed to 50° C. and was allowed to react for 13 hours while the solution was stirred. To this reaction liquid, 817.5 ml of water was added, and the mixture was cooled to 24.5° C. while stirred. Subsequently, the palladium-carbon was removed using a membrane filter, to obtain a homogeneous solution. The removed palladium-carbon was washed with 206 ml of water in total, and this washing water was mixed with the homogeneous solution previously obtained. To this mixed solution, 1328 ml of water was further added, and then 222.5 ml of a mixed solution of a 1 N aqueous solution of sodium hydroxide and ethanol mixed at a volume ratio of 3:1, was added to the mixture while stirred, to thereby adjust the solution to pH 8.31. This solution was stirred for about 1.5 hours at 25 to 28° C. to precipitate crystals, and then the solution was cooled to 3.1° C. over about 3 hours. Crystals precipitated therefrom were collected by filtration using a Kiriyama funnel. The wet crystals on the Kiriyama funnel were washed with 450 ml of water in total, and then these crystals were dried overnight under reduced pressure at 40° C. Thus, 30.56 g of the title compound was obtained.

### Reference Example 1b

### Synthesis of (R)—N-[3-[2-[2-(3-methylindazol-6-yloxy)ethylamino]-1-hydroxyethyl]phenyl]methanesulfonamide, “compound 1”

To 120.0 g (177.86 mmol) of (R)—N-benzyl-N-[3-[2-N′-benzyl-2-(1-benzyl-3-methylindazol-6-yloxy)ethylamino]-1-hydroxyethyl]phenyl]methanesulfonamide that can be produced according to a method described in WO 2003/035620, 24.1 g of 10% palladium-carbon (water content 52.07%, PE type manufactured by N.E. Chemcat Corp., Lot No. 217-044350) and 1200 ml of ethanol were added, and the system was purged with hydrogen. This solution was warmed to 59.6° C., and was allowed to react for about 4 hours while the solution was stirred. This reaction liquid was cooled to 34.9° C., and the system was purged with nitrogen. Subsequently, 31.8 ml of hydrochloric acid was added to the solution, and the system was purged again with hydrogen. This solution was warmed to 59.0° C., and was allowed to react for about 5 hours while the solution was stirred. The solution was cooled to 33.0° C., and the system was purged with nitrogen. 1000 ml of water was added to this reaction liquid, and then the palladium-carbon was removed using a membrane filter, to thereby obtain a homogeneous solution. The removed palladium-carbon was washed with 240 ml of water in total, and this washing water was mixed with the homogeneous solution previously obtained. To this mixed solution, 1990 ml of water was further added, and the mixture was warmed to 41.4° C. To the mixture, 369 ml of a 1 N aqueous solution of sodium hydroxide was added, and the mixture was adjusted to pH 8.33. This solution was stirred for about 2 hours at 35° C. to 40° C. to precipitate crystals, and then the solution was cooled to 5.3° C. over about 2 hours. The solution was stirred for about one hour while the temperature was maintained. Subsequently, crystals precipitated therefrom were collected by filtration using a Kiriyama funnel. The wet crystals on the Kiriyama funnel were washed with 240 ml of water in total, and then these crystals were dried overnight under reduced pressure at 40° C. Thus, 68.94 g of the title compound was obtained.

### Reference Example 2

### Synthesis of (R)—N-[3-[2-[2-(3-methylindazol-6-yloxy)ethylamino]-1-hydroxyethyl]phenyl]methanesulfonamide monohydrochloride, “monohydrochloride salt”

Thus, 40 ml of 2-propanol was added to 400.6 mg (0.99 mmol) of (R)—N-[3-[2-[2-(3-methylindazol-6-yloxy)ethylamino]-1-hydroxyethyl]phenyl]methanesulfonamide, and the mixture was heated to 70° C. to dissolve. To this solution, 0.090 ml of hydrochloric acid was added, and the mixture was naturally cooled while stirred, to thereby crystallize. After about 2 hours, this suspension reached 19.5° C., and crystals precipitated therefrom were collected by filtration and were dried under reduced pressure for about 3 hours at 40° C. Thus, 390.2 mg of the title compound was obtained.

### Example 1a

### Synthesis of (R)—N-[3-[2-[2-(3-methylindazol-6-yloxy)ethylamino]-1-hydroxyethyl]phenyl]methanesulfonamide dihydrochloride, “dihydrochloride salt”

To a mixture of 1.07 g (2.63 mmol) of (R)—N-[3-[2-[2-(3-methylindazol-6-yloxy)ethylamino]-1-hydroxyethyl]phenyl]methanesulfonamide, 10 ml of 2-propanol, and 3 ml of water, 473 μL (5.53 mmol, 2.1 equivalents) of concentrated hydrochloric acid was added, and the mixture was heated to 70° C. in an oil bath, to thereby obtain a homogeneous solution. While this temperature was maintained, 75 ml of 2-propanol was added dropwise over about 40 minutes, and thus crystals were precipitated. After completion of the dropwise addition, the solution was kept stirred for 10 minutes, and was left to stand for 1 hour 40 minutes, with the temperature of the oil bath set at 40° C. Crystals precipitated therefrom were collected by filtration and were dried under reduced pressure, and thus 1.08 g of the title compound was obtained.

### Example 1b

### Synthesis of (R)—N-[3-[2-[2-(3-methylindazol-6-yloxy)ethylamino]-1-hydroxyethyl]phenyl]methanesulfonamide dihydrochloride, “dihydrochloride salt”

To 1.01 g (2.48 mmol) of (R)—N-[3-[2-[2-(3-methylindazol-6-yloxy)ethylamino]-1-hydroxyethyl]phenyl]methanesulfonamide, 5.25 ml of 1 N hydrochloric acid was added, and the mixture was stirred at 25° C. to dissolve. While this solution was stirred at 25° C., 30 ml of 2-propanol was added dropwise thereto, and a trace amount of (R)—N-[3-[2-[2-(3-methylindazol-6-yloxy)ethylamino]-1-hydroxyethyl]phenyl]methanesulfonamide dihydrochloride was added as seed crystals. Thus, white crystals were precipitated. This suspension was cooled to 5.1° C., and then the crystals were collected by filtration and dried overnight under reduced pressure at 40° C. Thus, 882.2 mg of the title compound was obtained.

### Example 1c

### Synthesis of (R)—N-[3-[2-[2-(3-methylindazol-6-yloxy)ethylamino]-1-hydroxyethyl]phenyl]methanesulfonamide dihydrochloride, “dihydrochloride salt”

To 63.5 g (157.04 mmol) of (R)—N-[3-[2-[2-(3-methylindazol-6-yloxy)ethylamino]-1-hydroxyethyl]phenyl]methanesulfonamide, 165 ml of ethanol and 165 ml of 2 N hydrochloric acid were added, and the mixture was dissolved at 36.1° C. under stirring. The temperature of the dissolved liquid was adjusted to 30.2° C., and while the temperature was maintained at about 30° C., 1905 ml of ethanol was added dropwise thereto over about 2 hours under stirring, to thereby precipitate crystals. While the temperature was maintained, the solution was stirred for about 30 minutes, and then was cooled to 5.5° C. over about one hour. The solution was stirred for about one hour at or below 5° C., and then crystals were collected by filtration and were dried overnight under reduced pressure at 40° C. Thus, 55.77 g of the title compound was obtained.

### Test Example 1

### Test for Measuring Solubility

About 50 mg each of the “monohydrochloride salt” and the “dihydrochloride salt” were precisely weighed, and were each suspended in a solvent so as to obtain a concentration of 500 mg/ml, respectively. Each suspension was sonicated for 10 minutes, and then was shaken at room temperature for one hour. Subsequently, each of the supernatant of the suspension was subjected to centrifugal filtration (10° C., 3000 RPM, 20 minutes) using a centrifugal filter (Ultrafree-MC PTFE 0.2 μm, manufactured by Millipore, Inc.), to thereby prepare a saturated solution. Each of the saturated solutions was analyzed by HPLC, and the concentration of the saturated solution was calculated from the peak area. The results are presented in Table 1.

TABLE 1

| | Saturated solution concentration (mg/ml) | |
|---|---|---|
| | “monohydrochloride | |
| Solvent | salt” | “dihydrochloride salt” |
| Water | 36.7 | 189.3 |
| Physiological saline | 32.2 | 124.6 |
| pH 1.2 Buffer solution (1) | 73.5 | 127.5 |
| pH 6.8 Buffer solution (2) | 23.7 | 210.4 |

(1) 1^{st} fluid for dissolution test, pH 1.2 (Kanto Chemical Co., Inc., 11500-76)
(2) Phosphate buffer solution, pH 6.8 (Kanto Chemical Co., Inc., 33058-76)

From the results of Table 1, it was found that the “dihydrochloride salt” has significantly higher solubility compared with the “monohydrochloride salt”.

### Test Example 2

### Test for Measuring Solubility

The “dihydrochloride salt” was precisely weighed in the amounts indicated in Table 2, and 20 ml each of the solvent was added to the weighed samples. The samples were shaken for one hour at room temperature. After it was visually checked that the samples were not completely dissolved, the samples were centrifuged (10° C., 3000 RPM, 20 minutes), and the supernatant was filtered through a Membrane filter, to thereby produce saturated solutions. Each of the saturated solutions was analyzed by HPLC, and the concentration of the saturated solution was calculated from the peak area. The results are presented in Table 2.

TABLE 2

| | Amount | Saturated solution |
|---|---|---|
| Solvent | collected (g) | concentration (mg/ml) |
| Water | 5 | 143.209 |
| Physiological saline | 5 | 98.816 |
| pH 1.2 Buffer solution (1) | 4 | 97.772 |
| pH 4.0 Buffer solution (2) | 4 | 147.098 |
| pH 6.8 Buffer solution (3) | 4 | 155.951 |
| Ethanol | 0.1 | 0.516 |

(1) 1^{st} fluid for dissolution test, pH 1.2 (Kanto Chemical Co., Inc., 11500-76)
(2) Buffer solution of 0.05 mol/L acetic acid/sodium acetate, pH 4.0 (Kanto Chemical Co., Inc., 01901-76)
(3) 2^{nd} fluid for dissolution test, pH 6.8 (Kanto Chemical Co., Inc., 11499-84)

### Test Example 3

### Proton Nuclear Magnetic Resonance Spectrum (¹H-NMR)

Compound 1

The “compound 1” was dissolved in chloroform-d₁ (deuterated solvent) containing tetramethylsilane as an internal standard substance, and a nuclear magnetic resonance spectrum of the “compound 1” was measured under the following conditions:

Nuclear magnetic resonance apparatus: JNM AL300 (manufactured by JEOL, Ltd.)

Oscillation frequency: 300 MHz

Nuclide: ¹H

The “compound 1” exhibited peaks at δ (ppm): 2.42 (3H, s), 2.65-2.75 (2H, m), 2.9-2.95 (2H, m), 2.95 (3H, s), 4.05 (2H, t, J=5.50), 4.62 (1H, br), 5.36 (1H, br), 6.68 (1H, dd, J=8.80, 2.20), 6.82 (1H, d, J=1.83), 7.05-7.1 (2H, m), 7.2-7.3 (2H, m), 7.53 (1H, d, J=8.43), and 12.33 (1H, s). The ¹H-¹H correlation of those peaks supported the structure of the “compound 1.”

Here, in regard to the indication of the nuclear magnetic resonance spectrum data, J means the coupling constant (Hz), the symbols of the splitting pattern are such that s: singlet, d: doublet, t: triplet, dd: doublet doublet, m: multiplet, and br: broad. The same applies to the following.

Monohydrochloride Salt

Similarly, the “monohydrochloride salt” was dissolved in dimethyl sulfoxide-d₆ (deuterated solvent) containing tetramethylsilane as an internal standard substance, and a nuclear magnetic resonance spectrum of the “monohydrochloride salt” was measured under the following conditions:

Nuclear magnetic resonance apparatus: Gemini-300 (manufactured by Varian, Inc.)

Oscillation frequency: 300 MHz

Nuclide: ¹H

The “monohydrochloride salt” exhibited peaks at δ (ppm): 2.45 (3H, s), 3.00 (3H, s), 3.02-3.32 (2H, m), 3.42-3.50 (2H, m), 4.32-4.38 (2H, m), 4.79 (2H, br), 4.98-5.02 (1H, m), 6.78 (1H, dd, J=8.8, 2.2), 6.91 (1H, d, J=2.2), 7.12-7.17 (2H, m), 7.30-7.38 (2H, m), 7.61 (1H, d, J=8.8), 8.99 (1H, br), 9.26 (1H, br), and 9.85 (1H, s). The ¹H-¹H correlation of those peaks supported the structure of the “monohydrochloride salt”.

Dihydrochloride Salt

Similarly, the “dihydrochloride salt” was dissolved in dimethyl sulfoxide-d₆ (deuterated solvent) containing tetramethylsilane as an internal standard substance, and a nuclear magnetic resonance spectrum of the “dihydrochloride salt” was measured under the following conditions:

Nuclear magnetic resonance apparatus: am LA400 (manufactured by JEOL, Ltd.)

Oscillating frequency: 400 MHz

Nuclide: ¹H

The “dihydrochloride salt” exhibited peaks at δ (ppm): 2.52 (3H, s), 3.00 (3H, s), 3.10 (1H, br), 3.25 (1H, br), 3.47 (2H, br), 4.42 (2H, br), 5.06 (1H, d, J=10.0), 6.86 (1H, d, J=8.8), 6.96 (1H, s), 7.14 (1H, d, J=7.6), 7.18 (1H, d, J=8.0), 7.32 (1H, s), 7.34 (1H, t, J=8.2), 7.70 (1H, d, J=8.9), 9.18 (1H, br), 9.60 (1H, br), and 9.87 (1H, s). The ¹H-¹H correlation of those peaks supported the structure of the “dihydrochloride salt”.

### Test Example 4

### Mass Spectrum

A mass spectrum of the “dihydrochloride salt” was measured under the following conditions, and protonated molecules were detected at (m/z)=405. Thus, the results supported the structure of the “dihydrochloride salt”.

Conditions

Mass analysis apparatus: JMS-SX102 (manufactured by JEOL, Ltd.)

Ionization method: FAB

Detected ion: positive ion

Dissolving solvent: dimethyl sulfoxide

Matrix: m-nitrobenzyl alcohol

### Test Example 5

### Differential Scanning Calorimetric Analysis

Compound 1

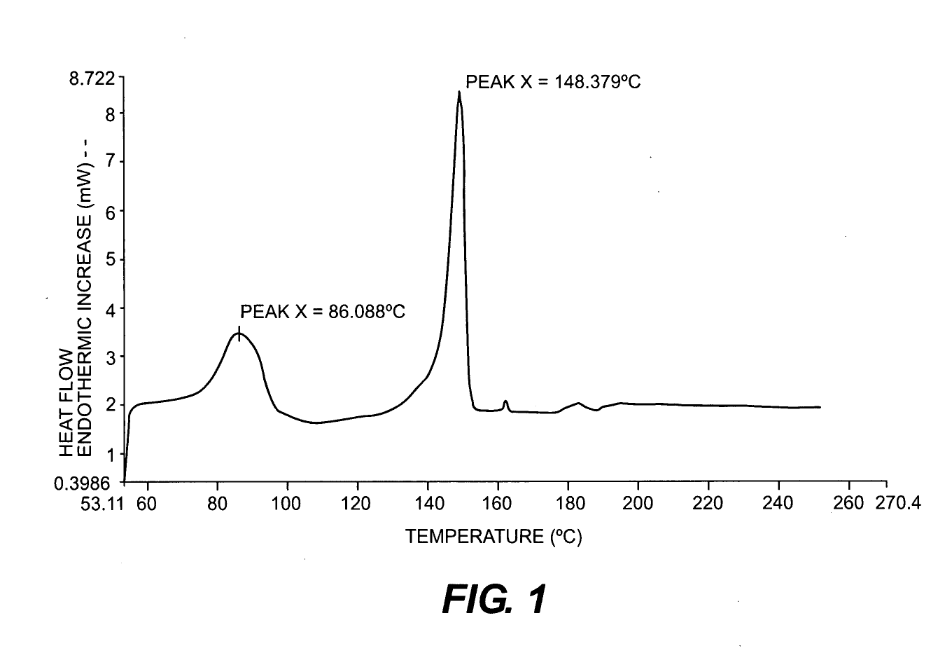

Differential scanning calorimetric analysis of the “compound 1” was carried out under the following conditions, and the spectrum as illustrated in FIG. 1 was obtained. An endothermic peak which was considered as a melting peak was recognized at 148° C.

Conditions

Calorimetric analyzing system: PYRIS Diamond DSC (manufactured by PerkinElmer, Inc.)

Temperature elevation condition: The temperature was elevated from 50° C. to 250° C. at a rate of 10° C. per minute.

Monohydrochloride Salt

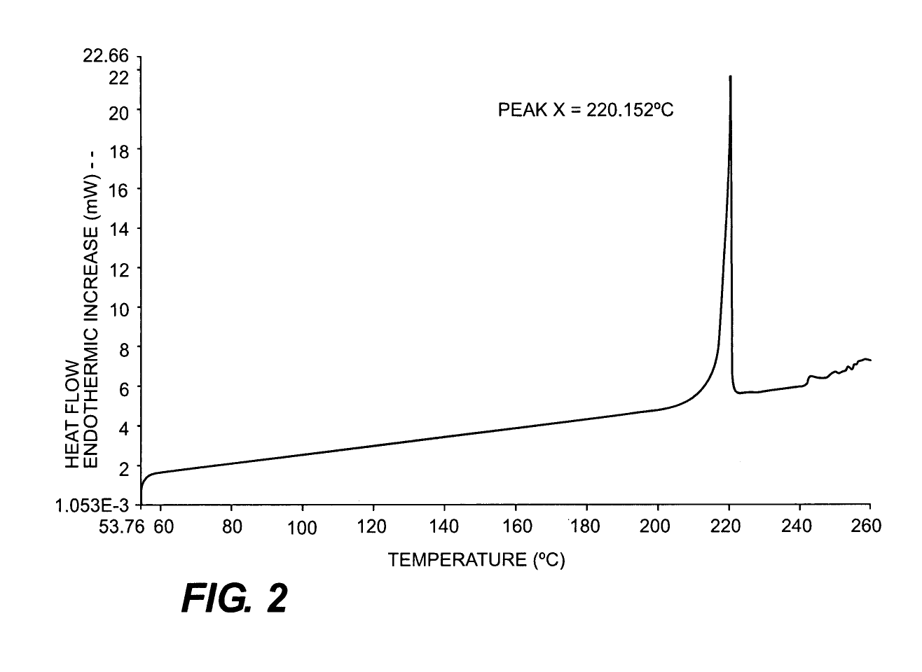

Differential scanning calorimetric analysis of the “monohydrochloride salt” was carried out under the following conditions, and the spectrum as illustrated in FIG. 2 was obtained. An endothermic peak which was considered as a melting peak was recognized at 220° C.

Conditions

Calorimetric analyzing system: PYRIS Diamond DSC (manufactured by PerkinElmer, Inc.)

Temperature elevation condition: The temperature was elevated from 50° C. to 260° C. at a rate of 10° C. per minute.

Dihydrochloride Salt

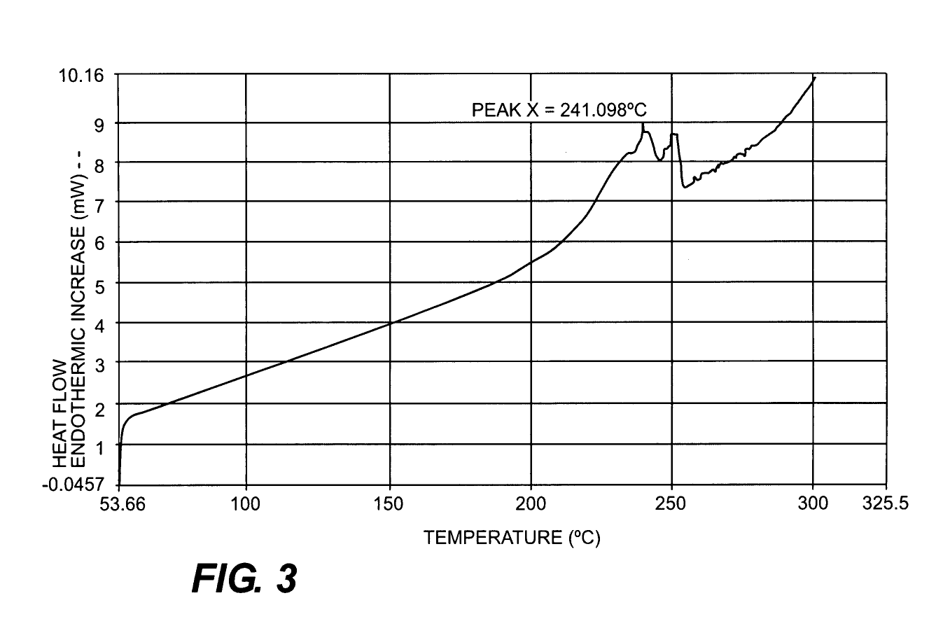

Differential scanning calorimetric analysis of the “dihydrochloride salt” was carried out under the following conditions, and the spectrum as illustrated in FIG. 3 was obtained. An endothermic peak which was considered as a decomposition peak was recognized at 241° C.

Conditions

Calorimetric analyzing system: PYRIS Diamond DSC (manufactured by PerkinElmer, Inc.)

Temperature elevation condition: The temperature was elevated from 50° C. to 300° C. at a rate of 10° C. per minute.

### Test Example 6

### Ion Exchange Chromatography

The salt number of chloride of the “monohydrochloride salt” was identified under the following ion exchange chromatography conditions, and 1.0 chloride ion per molecule of (R)—N-[3-[2-[2-(3-methylindazol-6-yloxy)ethylamino]-1-hydroxyethyl]phenyl]methanesulfonamide was recognized. Thus, it was confirmed that this substance is a salt formed by adding one hydrochloride molecule to (R)—N-[3-[2-[2-(3-methylindazol-6-yloxy)ethylamino]-1-hydroxyethyl]phenyl]methanesulfonamide.

Ion exchange chromatography conditions:

Sample concentration: 100 μg/ml

Ion chromatograph: Dionex DX-500 (manufactured by Dionex Corp.)

Detector: electrical conductivity detector

Column: DIONEX IonPac AS14, internal diameter 4 mm, length 25 cm

Guard column: DIONEX IonPac AG14, internal diameter 4 mm, length 5 cm

Column temperature: 30° C.

Mobile phase: 1.0 mmol/l aqueous solution of sodium hydrogen carbonate containing 3.5 mmol/l sodium carbonate

Flow rate: about 1.2 ml/min

Amount of injection: 10 μl.

Suppressor: ASRS-ULTRA (recycle mode: SRS 50 mA)

Dihydrochloride Salt

The salt number of chloride of the “dihydrochloride salt” was identified under the same ion exchange chromatography conditions as those used in the measurement of the “monohydrochloride salt”, and 2.0 chloride ions per molecule of (R)—N-[3-[2-[2-(3-methylindazol-6-yloxy)ethylamino]-1-hydroxyethyl]phenyl]methanesulfonamide was recognized. Thus, it was confirmed that this substance is a salt formed by adding two hydrochloride molecules to (R)—N-[3-[2-[2-(3-methylindazol-6-yloxy)ethylamino]-1-hydroxyethyl]phenyl]methanesulfonamide.

### Test Example 7

### Powder X-Ray Analysis

Compound 1

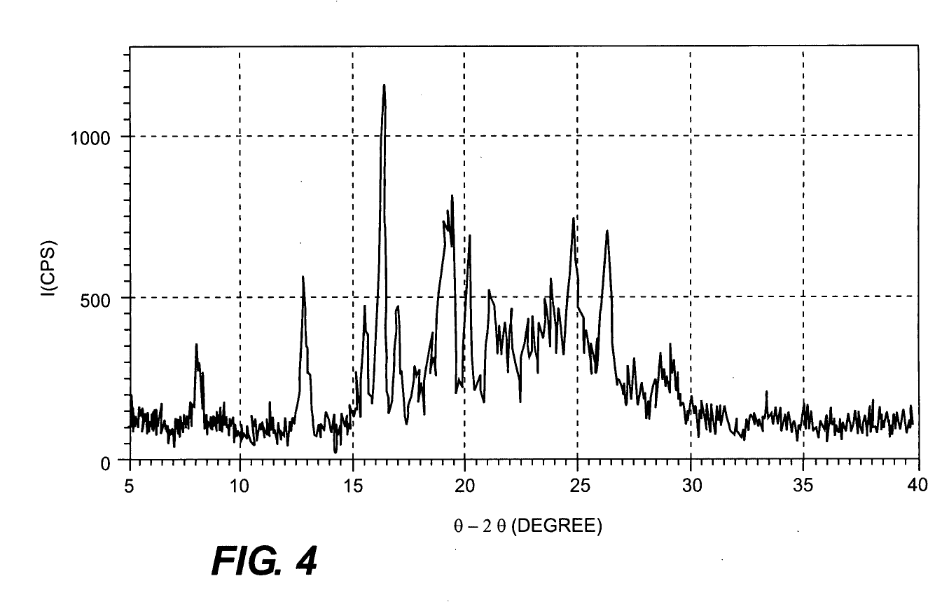

The “compound 1” was subjected to powder X-ray analysis under the following Conditions, and a diffraction spectrum as illustrated in FIG. 4 was obtained. In this powder X-ray diffraction spectrum, main peaks were recognized at 2θ value of 12.9°, 16.3°, 19.2° and 24.8°. Among these, peaks at 2θ value of 12.9°, 16.3° and 24.8° were characteristic peaks. Peaks were also recognized at 29 value of 15.6°, 17.0°, 20.2°, 21.2° and 26.4°, and any one or more peaks at 2θ value among 15.6°, 17.0° and 21.2° were also considered as peaks that are characteristic to the “compound 1.” Peaks were also recognized at 2θ value of 8.1°, 16.0°, 18.5°, 23.9°, 28.8° and 29.3°, and any one or more peaks at 2θ value among 8.1°, 16.0°, 18.5°, 28.8° and 29.3° can be considered as peaks that are characteristic to the “compound 1.”

It was judged even from a visual observation of the shape that the “compound 1” was in the form of crystals, and it was confirmed by the powder X-ray diffraction analysis that the “compound 1” was in the form of crystals.

Measurement Conditions

X-ray analysis apparatus: XRD-6000 (manufactured by Shimadzu Corp.)

X-ray source: CuKa (40 kV, 30 mA)

Operation mode: continuous

Scanning rate: 2°/min

Scanning drive axis: 0 to 20

Scanning range: 5° to 40°

Scattering slit: 1°

Light receiving slit: 0.15 mm

Monohydrochloride Salt

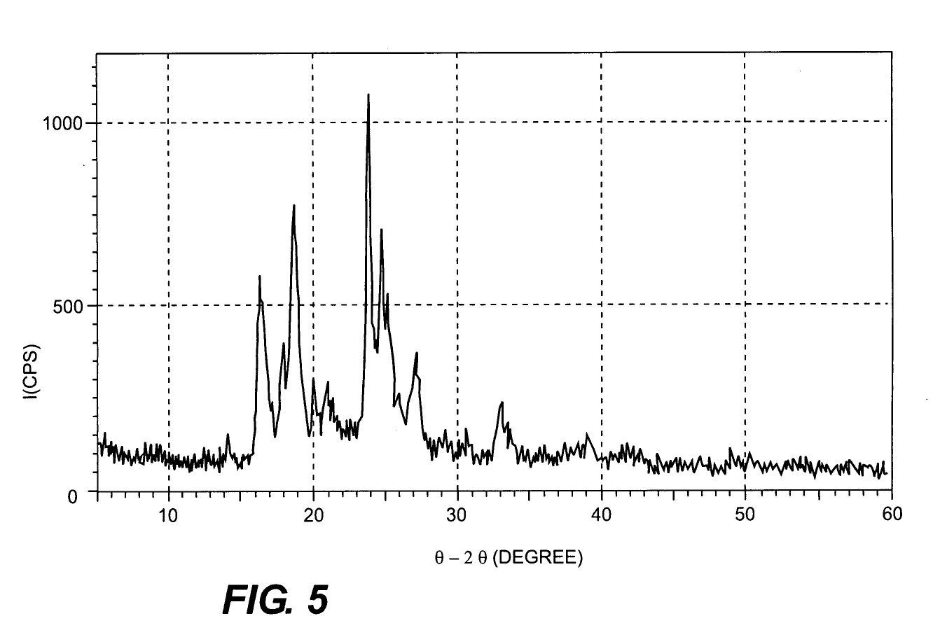

The “monohydrochloride salt” was subjected to powder X-ray analysis under the following conditions, and a diffraction spectrum as illustrated in FIG. 5 was obtained. In this powder X-ray diffraction spectrum, main peaks were recognized at 2θ value of 16.5°, 18.0°, 18.7°, 23.9°, 24.9° and 25.4°. Among these, peaks at 2θ value of 16.5°, 18.7°, 24.9° and 25.4° were characteristic peaks. Peaks were also recognized at 2θ value of 19.2°, 26.1°, 27.2° and 33.1°, and any one or more peaks at 2θ value among 26.1° and 33.1° were also considered as peaks that are characteristic to the “monohydrochloride salt”. Peaks were also recognized at 2θ value of 14.2°, 17.2°, 20.2°, 21.0°, 26.8° and 33.6°, and any one or more peaks at 2θ value among 14.2°, 17.2°, 21.0°, 26.8° and 33.6° can be considered as peaks that are characteristic to the “monohydrochloride salt”.

It was determined even from a visual observation of the shape that the “monohydrochloride salt” was in the form of crystals, and it was confirmed by the powder X-ray diffraction analysis that the “monohydrochloride salt” was in the form of crystals.

Measurement Conditions

X-ray analysis apparatus: XRD-6000 (manufactured by Shimadzu Corp.)

X-ray source: CuKa (40 kV, 30 mA)

Operation mode: continuous

Scanning rate: 2°/min

Scanning drive axis: 0 to 20

Scanning range: 5° to 60°

Scattering slit: 1°

Light receiving slit: 0.15 mm

Dihydrochloride Salt

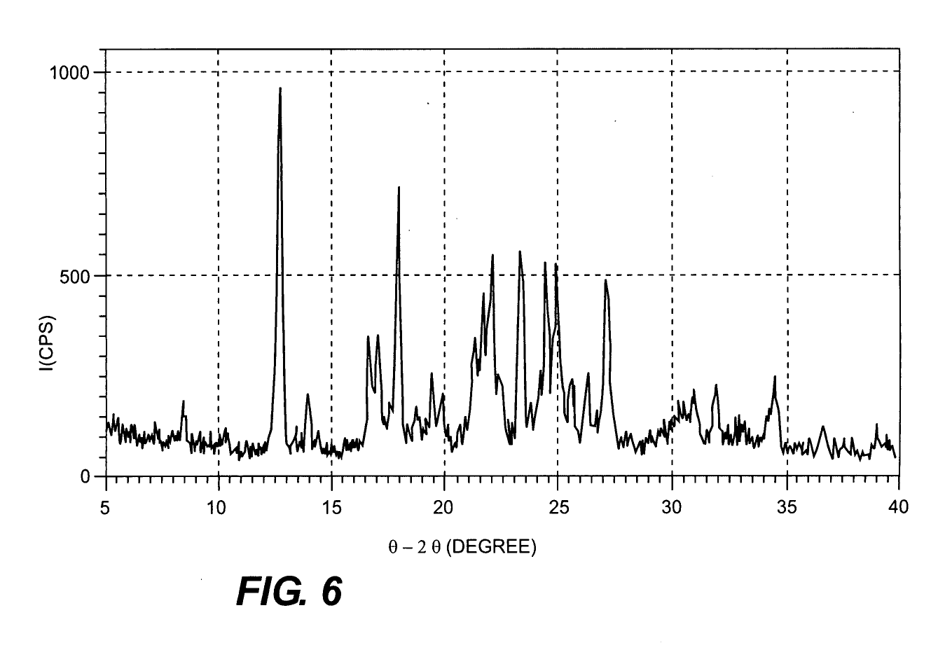

The “dihydrochloride salt” was subjected to powder X-ray analysis under the following conditions, and a diffraction spectrum as illustrated in FIG. 6 was obtained. In this powder X-ray diffraction spectrum, main peaks were recognized at 2θ value of 12.8°, 18.0°, 21.8° and 25.0°. Among these, peaks at 2θ value of 12.8°, 21.8° and 25.0° were characteristic peaks. Peaks were also recognized at 2θ value of 21.4°, 22.2°, 23.5°, 24.6° and 27.2°, and any one or more peaks at 2θ value among 21.4°, 22.2°, 23.5° and 24.6° were also considered as peaks that are characteristic to the “dihydrochloride salt”. Peaks were also recognized at 2θ value of 16.8°, 17.1°, 22.5°, 26.4°, 31.1° and 34.6°, and any one or more peaks at 2θ value among 16.8°, 17.1°, 22.5°, 31.1° and 34.6° can be considered as peaks that are characteristic to the “dihydrochloride salt”.

It was judged even from a visual observation of the shape that the “dihydrochloride salt” was in the form of crystals, and it was confirmed by the powder X-ray diffraction analysis that the “dihydrochloride salt” was in the form of crystals.

Measurement Conditions

X-ray analysis apparatus: XRD-6000 (manufactured by Shimadzu Corp.)

X-ray source: CuKα (40 kV, 30 mA)

Operation mode: continuous

Scanning rate: 2°/min

Scanning drive axis: θ to 20

Scanning range: 5° to 40°

Scattering slit: 1°

Light receiving slit: 0.15 mm

### Test Example 8

### Infrared Absorption Spectrum Compound 1

The “compound 1” was subjected to the measurement of an infrared absorption spectrum under the following conditions:

Measurement Conditions

Infrared spectrophotometer: FTIR-8300 (manufactured by Shimadzu Corp.)

Measurement method: potassium bromide tablet method

Control: potassium bromide tablet

Gain: auto

Aperture: auto

Minimum wavenumber: 400 cm⁻¹

Maximum wavenumber: 4000 cm⁻¹

Number of integration: 45 times

Detector: standard

Apodization function: Happ-Genzel

Resolution: 2 cm⁻¹

Mirror speed: 2.8

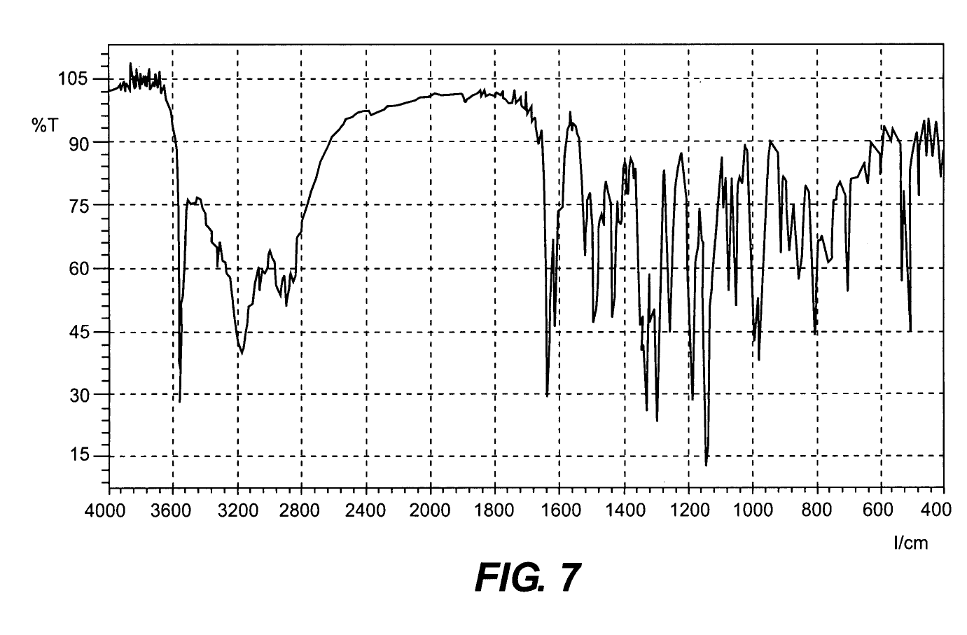

A spectrum as illustrated in FIG. 7 was obtained. In this infrared absorption spectrum, characteristic absorption peaks were recognized at 1329, 1295, 1145 and 1139 cm⁻¹. Absorption peaks were also recognized at 3552, 3541, 1633, 1185 and 974 cm⁻¹, and any one or more absorption peaks among these can be considered as characteristic absorption peaks of the “compound 1.”

Monohydrochloride Salt

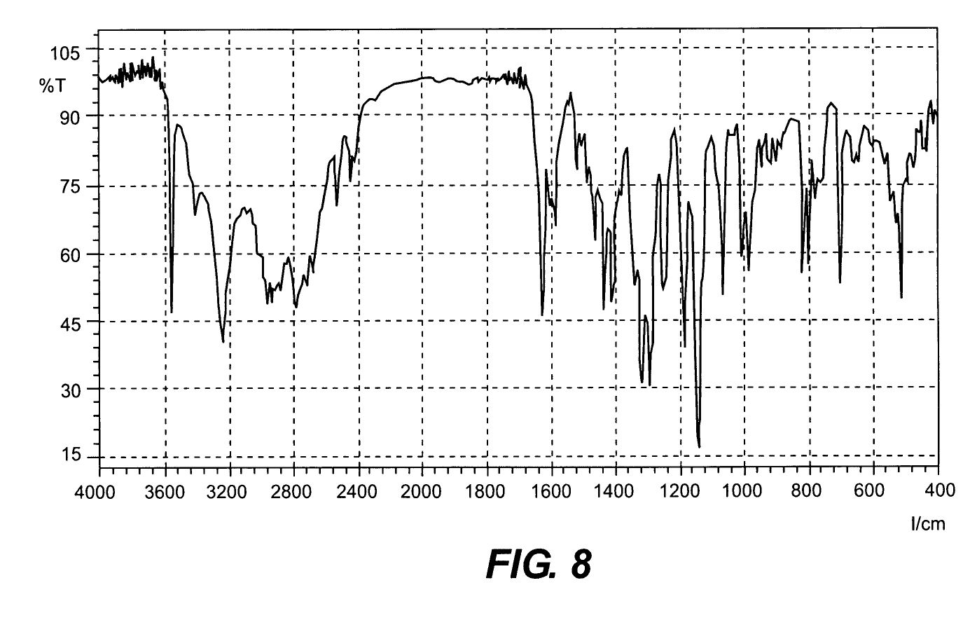

The “monohydrochloride salt” was subjected to the measurement of an infrared absorption spectrum under the same measurement conditions as those for the measurement of the “compound 1,” and a spectrum as illustrated in FIG. 8 was obtained. In this infrared absorption spectrum, characteristic absorption peaks were recognized at 1315, 1290, 1184 and 1144 cm⁻¹. Absorption peaks were also recognized at 3559, 3239, 2958, 2933, 2783, 1627, 1432, 1406 and 514 cm⁻¹, and any one or more absorption peaks among these can be considered as characteristic absorption peaks of the “monohydrochloride salt”.

Dihydrochloride Salt

The “dihydrochloride salt” was subjected to the measurement of an infrared absorption spectrum under the same measurement conditions as those for the measurement of the “compound 1,” and a spectrum as illustrated in FIG. 9 was obtained. In this infrared absorption spectrum, characteristic absorption peaks were observed at 1646, 1341, 1286 and 1150 cm⁻¹. Absorption peaks were also at 2970, 2778, 2718, 2664, 1591, 1408, 1201, 964 and 806 cm⁻¹, and any one or more absorption peaks among these can be considered as characteristic absorption peaks of the “dihydrochloride salt”.

### Test Example 9

### Blood Concentration Measurement Test (Calculation of Area Under Curve (AUC) in Drug Concentration-Time Curve)

The “monohydrochloride salt” and the “dihydrochloride salt” were respectively filled in an enteric capsule (Eudragit L coat, No. 5) to an amount of administration of 1 mg/kg in terms of the “compound 1” in a free form. The capsules were forcibly orally administered to male beagle dogs (Oriental Yeast Co., Ltd.) under fasting. 0.5, 1, 2, 3, 4, 6, 8, 10 and 24 hours after the administration, about 1 mL of blood was collected from the cephalic vein under non-anesthesia conditions, with a 2.5-mL syringe through which a heparin sodium solution had been passed through in advance (equipped with a 23 G injection needle). The collected blood was cooled in ice, and the blood plasma was separated by a centrifugal operation. After a lapse of 6 hours from the administration, the dogs were fed with a usual amount of food.

The concentration of the “compound 1” in a free form in the blood plasma was measured by the LC/MS/MS method in each case, and the AUC was calculated using the Non-Compartment model of WinNonlin software v5.0.1.

The results of Test Example 9 are presented in Table 3. (in the table, AUC_{inf} means the “AUC obtained when the blood plasma concentration was extrapolated from zero to the infinity”, mean is the “average”, SD is the “standard deviation”, and CV is the “coefficient of variation”.)

From these results, it was found that in the case of oral administration using enteric capsules, the CV (%) is smaller and the variation of the AUC is smaller in the case of administration of the “dihydrochloride salt” than in the case of administration of the “monohydrochloride salt”.

TABLE 3

| | AUC_{inf} (ng · hr/mL) | | |
|---|---|---|---|
| | mean | SD | CV (%) |
| “Monohydrochloride salt” | 1107.84 | 775.1 | 70.0 |
| “Dihydrochloride salt” | 1389.86 | 410.3 | 29.5 |

1. A crystal of (R)—N-[3-[2-[2-(3-methylindazol-6-yloxy)ethylamino]-1-hydroxyethyl]phenyl]methanesulfonamide dihydrochloride.

2. A crystal of (R)—N-[3-[2-[2-(3-methylindazol-6-yloxy)ethylamino]-1-hydroxyethyl]phenyl]methanesulfonamide dihydrochloride.

3. The crystal of (R)—N-[3-[2-[2-(3-methylindazol-6-yloxy)ethylamino]-1-hydroxyethyl]phenyl]methanesulfonamide dihydrochloride according to claim 2, wherein said crystal exhibits one or more major peaks at 2θ value selected from the group consisting of approximately 12.8°, 21.8° and 25.0° in the powder X-ray diffraction spectrum.

4. The crystal of (R)—N-[3-[2-[2-(3-methylindazol-6-yloxy)ethylamino]-1-hydroxyethyl]phenyl]methanesulfonamide dihydrochloride according to claim 2 wherein said crystal exhibits major peaks at 2θ value of approximately 12.8°, 18.0°, 21.8° and 25.0° in the powder X-ray diffraction spectrum.

5. The crystal of (R)—N-[3-[2-[2-(3-methylindazol-6-yloxy)ethylamino]-1-hydroxyethyl]phenyl]methanesulfonamide dihydrochloride according to claim 2, wherein said crystal exhibits major absorption peaks around wavenumbers of 1646, 1341, 1286 and 1150 cm⁻¹ in the infrared absorption spectrum.

6. The crystal of (R)—N-[3-[2-[2-(3-methylindazol-6-yloxy)ethylamino]-1-hydroxyethyl]phenyl]methanesulfonamide dihydrochloride according to claim 2, wherein said crystal exhibits a decomposition peak at approximately 241° C. in a differential scanning calorimetric analysis (heating rate: 10° C./min).

7. A method for producing the crystal of (R)—N-[3-[2-[2-(3-methylindazol-6-yloxy)ethylamino]-1-hydroxyethyl]phenyl]methanesulfonamide dihydrochloride according to claim 2, the method comprising:
preparing a solution by dissolving (R)—N-[3-[2-[2-(3-methylindazol-6-yloxy)ethylamino]-1-hydroxyethyl]phenyl]methanesulfonamide in a hydrochloric acid-containing solvent; and
isolating crystals precipitated from the solution after, if necessary, mixing the solution with a poor solvent of certain type.

8. A crystal of (R)—N-[3-[2-[2-(3-methylindazol-6-yloxy)ethylamino]-1-hydroxyethyl]phenyl]methanesulfonamide dihydrochloride which can be produced by the method for production according to claim 7.

9. A pharmaceutical composition comprising as an active ingredient the dihydrochloride salt according to claim 1.

10. A pharmaceutical composition comprising as an active ingredient the crystal according to claim 2.

11. A method for treating overactive bladder, comprising administering to a patient in need thereof a therapeutically effective amount of the dihydrochloride salt according to claim 1.

12. A method for treating overactive bladder, comprising administering to a patient in need thereof a therapeutically effective amount of the crystal according to claim 2.

13. Use of the dihydrochloride salt according to claim 1, for manufacturing a therapeutic agent for overactive bladder.

14. Use of the crystal according to claim 2, for manufacturing a therapeutic agent for overactive bladder.

15. The crystal of (R)—N-[3-[2-[2-(3-methylindazol-6-yloxy)ethylamino]-1-hydroxyethyl]phenyl]methanesulfonamide dihydrochloride according to claim 3, wherein said crystal exhibits major peaks at 2θ value of approximately 12.8°, 18.0°, 21.8° and 25.0° in the powder X-ray diffraction spectrum.

16. The crystal of (R)—N-[3-[2-[2-(3-methylindazol-6-yloxy)ethylamino]-1-hydroxyethyl]phenyl]methanesulfonamide dihydrochloride according to claim 3, wherein said crystal exhibits major absorption peaks around wavenumbers of 1646, 1341, 1286 and 1150 cm⁻¹ in the infrared absorption spectrum.

17. The crystal of (R)—N-[3-[2-[2-(3-methylindazol-6-yloxy)ethylamino]-1-hydroxyethyl]phenyl]methanesulfonamide dihydrochloride according to claim 4, wherein said crystal exhibits major absorption peaks around wavenumbers of 1646, 1341, 1286 and 1150 cm⁻¹ in the infrared absorption spectrum.

18. The crystal of (R)—N-[3-[2-[2-(3-methylindazol-6-yloxy)ethylamino]-1-hydroxyethyl]phenyl]methanesulfonamide dihydrochloride according to claim 3, wherein said crystal exhibits a decomposition peak at approximately 241° C. in a differential scanning calorimetric analysis (heating rate: 10° C./min).

19. The crystal of (R)—N-[3-[2-[2-(3-methylindazol-6-yloxy)ethylamino]-1-hydroxyethyl]phenyl]methanesulfonamide dihydrochloride according to claim 4, wherein said crystal exhibits a decomposition peak at approximately 241° C. in a differential scanning calorimetric analysis (heating rate: 10° C./min).

20. The crystal of (R)—N-[3-[2-[2-(3-methylindazol-6-yloxy)ethylamino]-1-hydroxyethyl]phenyl]methanesulfonamide dihydrochloride according to claim 5, wherein said crystal exhibits a decomposition peak at approximately 241° C. in a differential scanning calorimetric analysis (heating rate: 10° C./min).
